(19) United States
(12) Patent Application Publication
(10) Pub. No.: US 2006/0134751 A1
(43) Pub. Date: Jun. 22, 2006

(54) Identification of novel MS4A gene family members expressed by hematopoietic cells

(76) Inventors: Thomas F. Tedder, Durham, NC (US); Ying Hua Liang, Durharm, NC (US)

Correspondence Address: FISH & NEAVE IP GROUP;ROPES & GRAY LLP, ONE INTERNATIONAL PLACE, BOSTON, MA, 02110-2624, US

(21) Appl. No.: 11/347,766

(22) Filed: Feb. 2, 2006

Related U.S. Application Data
(63) Continuation of application No. 10/433,287, filed on Sep. 30, 2003, now abandoned.
(60) Provisional application No. 60/254,362, filed on Dec. 8, 2000.
(60) Provisional application No. 60/270,057, filed on Feb. 20, 2001.

(51) Int. Cl. C07K 14/705; C07K 16/28; C07H 21/04; C12P 21/06

(52) U.S. Cl. 435/69.1; 536/23.5; 530/350; 530/388.22; 435/320.1; 435/325

(57) ABSTRACT

Isolated nucleic acids encoding MS4A polypeptides, isolated MS4A polypeptides, and uses thereof. The disclosed MS4A nucleic acids and polypeptides can be used to generate a mouse model of atopic disorders, for drug discovery screens, and for therapeutic treatment of atopic disorders or other MS4A-related conditions.

### CROSS REFERENCE TO RELATED APPLICATIONS

This application is based on and claims priority to U.S. Provisional Application Ser. No. 60/254,362, filed Dec. 8, 2000, and U.S. Provisional Application Ser. No. 60/270,057 filed Feb. 20, 2001, herein incorporated by reference in their entirety.

### GRANT STATEMENT

This work was supported by NIH grants CA-81776 and CA-54464. Thus, the U.S. Government has rights in the invention.

### FIELD OF THE INVENTION

The present invention generally relates to a new class of MS4A proteins characterized by a membrane-embedded structure. More particularly, the present invention provides MS4A nucleic acid and polypeptide sequences, chimeric genes comprising disclosed MS4A sequences, antibodies that specifically recognize MS4A polypeptides, and uses thereof.

Table of Abbreviations

| | |
|---|---|
| ATCC | American Tissue Culture Collection |
| CD20 | CD20 B lymphocyte differentiation antigen |
| FcεRlβ | high-affinity IgE receptor β chain |
| GFP | green fluorescent protein |
| htgs | GenBank human genomic database |
| HTm4 | hematopoietic CD20-like antigen |
| MS4A family | membrane spanning 4-domain family, |
| | subfamily A |

### BACKGROUND ART

CD20, FcεRIβ, and HTm4 are three cell surface proteins expressed by hematopoietic cells that represent members of a nascent gene family (Adra et al. (1999) Clin Genet 55:431437, Kinet (1999) Annu Rev Immunol 17:931-972; Tedder and Engel (1994) Immunol Today 15:450-454). The deduced amino acid sequence of human and mouse CD20 first demonstrated a cell surface protein containing four membrane-spanning regions, N- and C-terminal cytoplasmic domains, and an ˜50 amino acid loop that serves as the extracellular domain (Einfeld et al. (1988) EMBO J 7:711-717; Stamenkovic and Seed (1988) J Exp Med 167:1975-1980; Tedder et al. (1988a) J Immunol 141:4388-4394; Tedder et al. (1988b) Proc Natl Acad Sci USA 85:208-212). Human CD20 shares 20% amino acid sequence identity with FcεRIβ and HTm4 (Adra et al. (1994) Proc Natl Acad Sci USA 91:10178-10182, Küster et al. (1992) J Biol Chem 267:12782-12787). Moreover, these three proteins have a similar overall structure in man, mouse, and rat with significant sequence identity within the first three membrane-spanning domains (Kinet et al. (1988) Proc Natl Acad Sci USA 85:6483-6487; Ra et al. (1989) Nature 19:1771-7; Tedder et al., 1988a). In addition, all three genes are located in the same region of human chromosome 11q12-13.1 (Adra et al., 1994; Hupp et al. (1989) J Immunol 143:3787-3791; Tedder et al. (1989a) J Immunol 142:2555-2559) and mouse chromosome 19 (Hupp et al. 1989; Tedder et al., 1988a). These three genes are therefore likely to have evolved from a common precursor.

Despite structural and sequence conservation between CD20, FCεRIβ and HTm4, transcription of each gene is differentially regulated. CD20 is only expressed by B lymphocytes (Stashenko et al. (1980) J Immunol 125:1678-1685; Tedder et al., 1988a). FcεRIβ is expressed by mast cells and basophils (Kinet, 1999). HTm4 is expressed by diverse lymphoid and myeloid origin hematopoietic cells (Adra et al., 1994). Although the function of HTm4 remains unexplored, CD20 and FcεRIβ have critical roles in cell signaling. CD20 forms a homo- or hetero-tetrameric complex that is functionally important for regulating cell cycle progression and signal transduction in B lymphocytes (Tedder and Engel, 1994). CD20 additionally regulates transmembrane Ca⁺⁺ conductance, possibly as a functional component of a Ca⁺⁺-permeable cation channel (Bubien et al. J Cell Biol 121:1121-1132; Kanzaki et al. (1997a) J Biol Chem 272:14733-14739; Kanzaki et al. (1997b) J Biol Chem 272:4964-4969; Kanzaki et al. (1995) J Biol Chem 270:13099-13104). FcεRIRβ is part of a tetrameric receptor complex consisting of α, β, and two γ chains (Blank et al. (1989) Nature 337:187-189). FcεRIβ mediates interactions with IgE-bound antigens that lead to cellular responses such as the degranulation of mast cells. Specifically, the FcεRIβ subunit functions as an amplifier of FcεRIβ-mediated activation signals (Dombrowicz et al. (1998) Immunity 8:517-529; Lin et al. (1996) Cell 85:985-995). Because of their unique structure and sequence homology, CD20, FcεRIβ, and HTm4 are likely to share overlapping functional properties.

CD20 and FcεRIβ are also important clinically. Antibodies against CD20 are effective in treating non-Hodgkin's lymphoma (McLaughlin et al. (1998) Oncology 12:1763-1769; Onrust et al. (1989) J Biol Chem 264:15323-15327; Weiner (1999) Semin Oncol 26:43-51). Genetic variations at chromosome 11q12-13 can also play a role in the pathogenesis of allergic diseases (Adra et al., 1999; Kinet, 1999). Recent studies suggest that FcεRIβ contributes to such diseases, and other genetic elements in this region likely also contribute to allergic disease.

Since CD20, FcεRIβ, and HTm4 are likely to have evolved by duplication of an ancestral gene, other related proteins might exist that form additional receptor complexes. In view of the clinical importance noted above, the identification of such proteins thus represents a long-felt and ongoing need in the art. To address this need, applicants have identified novel human and mouse proteins that span the cell membrane at least four times and share high levels of amino acid sequence identity with CD20, FcεRIβ, and HTm4. This finding reveals a new gene family that has been designated herein as the MS4A family (membrane spanning 4-domain family, subfamily A). Currently this family contains at least 10 subgroups (MS4A1 through MS4A12) that encode at least 21 previously unidentified human and mouse proteins expressed by hematopoietic cells and by diverse cell types in non-hematopoietic tissues.

### SUMMARY OF THE INVENTION

The present invention discloses isolated MS4A polypeptides and isolated nucleic acid molecules encoding the same. Preferably, an isolated MS4A polypeptide, or functional portion thereof, comprises a polypeptide encoded by the nucleic acid molecule of any one of the odd numbered SEQ ID NOs:1-37 a polypeptide encoded by a nucleic acid molecule that is substantially identical to any one of the odd-numbered SEQ ID NOs:1-37, a polypeptide fragment encoded by a 20 nucleotide sequence that is identical to a contiguous 20 nucleotide sequence of any one of the odd-numbered SEQ ID NOs:1-37, a polypeptide having an amino acid sequence of any one of the even-numbered SEQ ID NOs:2-38, a polypeptide that is a biological equivalent of any one of the even-numbered SEQ ID NOs:2-38, or a polypeptide that is immunologically cross-reactive with an antibody that shows specific binding with a polypeptide comprising some or all amino acids of any one of the even-numbered SEQ ID NOs:2-38.

The present invention further teaches chimeric genes having a heterologous promoter that drives expression of a nucleic acid sequence encoding a MS4A polypeptide. Preferably, the chimeric gene is carried in a vector and introduced into a host cell so that a MS4A polypeptide of the present invention is produced. Preferred host cells include but are not limited to a bacterial cell, a hamster cell, a mouse cell, or a human cell.

In another aspect of the invention, a method is provided for detecting a nucleic acid molecule that encodes a MS4A polypeptide. According to the method, a biological sample having nucleic acid material is hybridized under stringent hybridization conditions to a MS4A nucleic acid molecule of the present invention. Such hybridization enables a nucleic acid molecule of the biological sample and the MS4A nucleic acid molecule to form a detectable duplex structure. Preferably, the MS4A nucleic acid molecule includes some or all nucleotides of any one of the odd-numbered SEQ ID NOs:1-37. Also preferably, the biological sample comprises human nucleic acid material.

The present invention further teaches an antibody that specifically recognizes a MS4A polypeptide. Preferably, the antibody recognizes some or all amino acids of any one of the even-numbered SEQ ID NOs:2-38. A method for producing a MS4A antibody is also disclosed, and the method comprises recombinantly or synthetically producing a MS4A polypeptide, or portion thereof; formulating the MS4A polypeptide so that it is an effective immunogen; immunizing an animal with the formulated polypeptide to generate an immune response that includes production of MS4A antibodies; and collecting blood serum from the immunized animal containing antibodies that specifically recognize a MS4A polypeptide. Antibody-producing cells can be optionally fused with an immortal cell line whereby a monoclonal antibody that specifically recognizes a MS4A polypeptide can be selected. Preferably, the MS4A polypeptide used as an immunogen includes some or all amino acid sequences of any one the even-numbered SEQ ID NOs:2-38.

A method is also provided for detecting a level of MS4A polypeptide using an antibody that specifically recognizes a MS4A polypeptide. According to the method, a biological sample is obtained from an experimental subject and a control subject, and a MS4A polypeptide is detected in the sample by immunochemical reaction with the MS4A antibody. Preferably, the antibody recognizes amino acids of any one of the even-numbered SEQ ID NOs:2-38, and is prepared according to a method of the present invention for producing such an antibody.

The present invention further discloses a method for identifying a compound that modulates MS4A function. The method comprises: exposing an isolated MS4A polypeptide to one or more compounds, and assaying binding of a compound to the isolated MS4A polypeptide. A compound is selected that demonstrates specific binding to the isolated MS4A polypeptide. Preferably, the MS4A polypeptide used in the binding assay of the method includes some or all amino acids of any one of the even-numbered SEQ ID NOs:2-38.

Also provided is a method for identifying a regulator of MS4A gene expression. The method comprises (a) exposing a cell sample with a candidate compound to be tested, the cell sample containing at least one cell containing a DNA construct comprising a modulatable transcriptional regulatory sequence of a MS4A-encoding nucleic acid and a reporter gene which is capable of producing a detectable signal; (b) evaluating an amount of signal produced in relation to a control sample; and (c) identifying a candidate compound as a modulator of MS4A gene expression based on the amount of signal produced in relation to a control sample. Preferably, the modulatable transcriptional regulatory sequence of a MS4A-encoding nucleic acid comprises a sequence that is immediately upstream of the initial coding region of a MS4A gene as set forth in any one of SEQ ID NOs:73-81.

The present invention further provides a method for modulating MS4A function in a subject. According to the method, a pharmaceutical composition is prepared that includes a substance capable of modulating MS4A expression or function, and a carrier. An effective dose of the pharmaceutical composition is administered to a subject, whereby MS4A activity is altered in the subject. Provided are therapeutic methods wherein a change in MS4A activity comprises a shift in the abundance of cell subpopulations expressing said protein, modulation of [Ca²⁺]ᵢ levels, or altered cell function. In a preferred embodiment, the substance used to perform this method shows specific binding to some or all amino acids of any one of the even-numbered SEQ ID NOs:2-38, and was discovered by a method of the present invention. In another embodiment, MS4A function is disrupted by immunizing a subject with an effective dose of the disclosed MS4A polypeptide. The immune system of the subject produces an antibody that specifically recognizes the MS4A polypeptide, and preferably recognizes some or all of amino acids of any one of the even-numbered SEQ ID NOs:2-38. In a further embodiment, a gene therapy vector is used, the vector comprising a nucleotide sequence encoding a MS4A polypeptide. Alternatively, the gene therapy vector comprises a nucleotide sequence encoding a nucleic acid molecule, a peptide, or a protein that interacts with a MS4A nucleic acid or polypeptide. Preferably, the subject is a human subject.

Accordingly, it is an object of the present invention to provide novel MS4A nucleic acid and polypeptide sequences, and novel methods relating thereto. This object is achieved in whole or in part by the present invention.

An object of the invention having been stated above, other objects and advantages of the present invention will become apparent to those skilled in the art after a study of the following description of the invention, Figures and non-limiting Examples.

### BRIEF DESCRIPTION OF THE DRAWINGS

FIG. 1 depicts cDNAs encoded by fifteen new human or mouse MS4A gene products. Consensus sequences from cDNAs and overlapping ESTs are indicated by their GenBank Accession numbers. Representative full-length cDNAs for each gene product are shown, except for MS4a3 which was not full-length. 5′ and 3′ untranslated sequences are shown as horizontal lines with relative nucleotide lengths shown. Coding regions are shown as boxes with translation initiation and termination codons and their relative nucleotide locations shown. Poly(A) attachment signal sequences (AATAAA) are indicated when known. Deduced hydrophobic regions are shown as filled boxes with the predicted membrane-spanning domains shown as TM1-TM4. Additional hydrophobic regions in MS4A4 proteins are shown as shaded boxes. Sites of putative nucleotide polymorphisms in MS4A6A are indicated by two (X)s.

FIG. 2 depicts exon-intron organization of the human MS4A genes. The maps were constructed by aligning known and predicted MS4A cDNA sequences with human genomic sequences as described in Materials and Methods. Exons are shown as boxes with the predicted translation initiation codons (ATG), transmembrane domains (TM) and termination codons indicated on the top. All exon and intron distances are shown to scale. Gaps indicate where intron distances have not been determined for MS4A3, MS4A4A, and MS4A12. Two long introns present in MS4A4E are not to scale but the intron lengths are indicated. Exon numbering for MS4A1, and MS4A2 is as published (Kuster et al., 1992; Tedder et al., 1988a; Tedder et al., 1988b).

FIG. 3 shows human MS4A4E protein and transcript sequences predicted from genomic DNA sequences. MS4A4E sequences are compared with human MS4A4A cDNA (disclosed herein) and genomic sequences. Gaps were introduced to provide optimal alignment. The boxed MC sequence near the 5′ end of the MS4A4A sequence indicates the length of the most 5′ MS4A4A cDNA sequence. Sequences upstream of this are based on contiguous genomic DNA sequences. Nucleotide numbering is based on the MS4A4A cDNA sequence, disclosed herein. Predicted translation initiation codons are shaded. Predicted membrane-spanning regions are underlined. An asterisk indicates predicted translation termination codons. Potential poly-A attachment signal sequences (AATAAA) are boxed.

FIG. 4 shows human MS4A6E protein and transcript sequences predicted from genomic DNA and overlapping cDNA sequences. PredictedMS4A6E transcript sequences are compared with human MS4A6A cDNA sequence (disclosed herein). Gaps were introduced in the nucleotide sequence to provide optimal alignment. The 5′ end of both transcripts start at 3′ splice-acceptor sites which demark the first translated exons for both genes. The 5′ end of the putative MS4A6E transcript is based on genomic DNA sequence, while the predicted sequences starting at nucleotide 60 were based on both genomic DNA sequences and overlapping cDNA sequences. A gap in the MS4A6A sequence is indicated where TM1/2 and TM2 exons are not found in MS4A6E transcripts. MS4A6A nucleotide numbering is based on the cDNA sequence (disclosed herein). Predicted translation initiation codons are shaded. Predicted membrane-spanning regions are underlined. An asterisk indicates the predicted translation termination codon for the MS4A6E protein.

FIG. 5 shows human MS4A10 protein and transcript sequences predicted from human genomic DNA sequences. MS4A10 nucleotide sequence is compared with mouse MS4A10 cDNA sequence (disclosed herein). The 5′ end of both transcripts start at 3′ splice-acceptor sites which demark the first translated exons for both genes. MS4A10 nucleotide numbering is based on the cDNA sequence (disclosed herein). Predicted translation initiation codons are shaded. Predicted membrane-spanning regions are underlined. An asterisk indicates predicted translation termination codon for the MS4A10 protein. Potential poly-A attachment signal sequences (AATAAA) are boxed.

FIG. 6 depicts a physical linkage map for the MS4A genes. A scheme for chromosome 11 structure is shown on the left with the mapped locations for MS4A1, MS4A2 and MS4A3 indicated. Representative human BAC clones are shown as vertical black bars with clone names shown on the top and clone size shown at the bottom. All distances are shown to the indicated scale. The distance between and spatial relationship of RP11-312N17 to the four other overlapping BACs shown at the bottom are unknown. Thin bars indicate continuous characterized (mapped or sequenced) regions of DNA that contain identified MS4A genes. When the relative position of this region of DNA is known relative to the representative BACs that are shown, the thin bars overlay the BAC. The mapped position of each MS4A gene is indicated on the right with the relative direction of gene translation indicated by arrows (→). In some cases, approximate distances between MS4A genes (termination codons to the translation initiation codon for the next gene) are indicated in base pairs (bp). In some cases, approximate MS4A gene size is indicated showing the distance between predicted translation initiation codons and translation termination codons as show in FIG. 7.

FIG. 7 depicts deduced amino acid sequences for CD20 (human A1, SEQ ID-NO:40; mouse a1, SEQ ID NO:48), FcεRIβ (human A2, SEQ ID NO:42; mouse a2, SEQ ID NO:50), HTm4 (human A3, SEQ ID NO:44; mouse a3, SEQ ID NO:20), and 19 new MS4A (human) (even-numbered SEQ ID NOs:2-18, 46) and MS4a (mouse and pig) proteins (even-numbered SEQ ID NOs:22-38, 56). Gaps were introduced to optimize alignments. Numbers represent predicted residue positions. The predicted membrane-spanning regions (TM1-TM4) are indicated. Predicted intronlexon splice junctions are indicated by vertical bars where information was available. Amino acids common to 10 or more proteins are shaded. *indicates partial sequence for the MS4a3 protein. CD20, FcεRIβ, and HTm4 sequences and known intronlexon borders (SEQ ID NOs:39-44, 47-50) are as published (Adra et al., 1994; Kuster et al., 1992; Ra et al., 1989; Tedder et al., 1988a; Tedder et al., 1989b; Tedder et al., 1988b). MS4A12 represents a conceptual translation (SEQ ID NO:46) of a human colon mucosa cDNA sequence (GenBank AK000224, SEQ ID NO:45), and MS4a12 represents a conceptual translation (SEQ ID NO:56) of a homologous cDNA sequence from pig (GenBank AJ236932, SEQ ID NO:55).

FIG. 8 depicts UPGMA (unweighted pair group method using arithmetic averages) tree of deduced MS4A and MS4a protein sequences. Horizontal tree branch length is a measure of sequence relatedness. For example, MS4a4B and MS4a4C are the most similar in sequence, while CD20 (MS4A1) sequences were the most divergent from other family members. The MS4a12p sequence was from pig, while all other MS4a sequences were from mouse. The UPGMA tree was generated using Geneworks version 2.0 (IntelliGenetics, Inc., Mountain View, Calif., USA).

FIG. 9 shows immunofluorescent detection of CD20 expression during B cell development. Single cell suspensions of leukocytes were isolated from wild-type mice, stained using MB20-13 (visualized using a PE-conjugated, anti-mouse IgG3 antibody) and anti-B220 (FITC-conjugated) monoclonal antibodies, and examined by two-color immunofluorescence staining with flow cytometry analysis. Quadrant gates indicate negative and positive populations of cells as determined using isotype-matched control monoclonal antibodies. The gated cell populations correspond to the cells described in Table 7, and are shown for reference. These results are representative of those obtained with six (6) two month-old wild type mice.

FIG. 10 summarizes the strategy for targeted disruption of the mouse CD20 gene.

FIG. 10A shows genomic clones encoding CD20.

FIG. 10B shows the intron-exon organization of the wild typeCD20 allele containing exons 5-8 (shaded squares).

FIG. 10C shows the structure of the CD20 targeting vector.

FIG. 10D shows the predicted structure of the CD20 allele after gene targeting in ES cells by homologous recombination. The EcoR V restriction site in exon 6 is deleted as indicated.

FIG. 10E presents Southern blot analysis of tail DNA from two wild type and four CD20^{-/-} mice. Genomic DNA was digested with EcoR V, transferred to nitrocellulose and hybridized with the 5′ probe indicated in (D).

FIG. 10F shows PCR amplification of genomic DNA from wild type and CD20^{-/-} mice using primers that bind in exons 6 and 7. Amplification of glyceraldehyde-3-phosphate dehydrogenase (G3PDH) is shown as a positive control.

FIG. 10G shows PCR amplification of cDNA generated from splenic RNA of wild type and CD20^{-/-} mice. Each reaction mixture contained a sense primer that hybridized with sequences encoded by exon 3 and antisense primers that hybridized with either exon 6 or Neo^{r} gene promoter sequences.

FIGS. 10H and 10I show reactivity of the MB20-13 monoclonal antibody with CD20 cDNA-transfected (thick line) or untransfected (dashed line) 300.19 cells (FIG. 10H) or Chinese Hamster Ovary (CHO) cells (FIG. 10I). The thin lines represent CD20 cDNA-transfected cells stained with secondary antibody alone or an isotype-control monoclonal antibody. Indirect immunofluorescence staining was visualized by flow cytometry analysis.

FIG. 10J shows immunofluorescent staining of splenocytes from CD20^{-/-} or wild type mice with MB20-13 (visualized using a PE-conjugated, anti-mouse IgG3 antibody) and anti-B220 (FITC-conjugated) monoclonal antibodies. Splenocytes from CD20^{-/-} mice generated histograms identical to those obtained without MB20-1 monoclonal antibody present, using the secondary antibody alone.

FIG. 11 depicts immunofluorescent detection of B lymphocyte subpopulations in CD20^{-/-} and wild type mice. Lymphocytes were isolated and examined by two color immunofluorescent staining with flow cytometry analysis. Quadrants delineated by squares indicate negative and positive populations of cells as determined using unreactive monoclonal antibody controls. The gated cell populations correspond to the cells described in Table 7 that represent at least 6 mice of each genotype.

FIG. 12 shows altered signal transduction in CD20^{-/-} B cells. FIG. 12 also shows CD19 expression by splenocytes from CD20^{-/-} (thin line) and wild type (thick line) mice. Immunofluorescence staining using PE-conjugated anti-CD19 monoclonal antibody with flow cytometry analysis. The dashed line represents staining of wild type splenocytes with a control antibody.

FIG. 12A presents calcium responses induced by BCR or CD19 ligation in CD20^{-/-} and wild type B cells. Splenocytes were loaded with 1 μM indo-1-AM ester and B cells were stained with FITC-conjugated anti-B220 antibody. At 1 min (arrow), optimal concentrations of goat anti-IgM F(ab′)₂ antibody fragments, anti-CD19 monoclonal antibody or Thapsigargin were added, with or without EGTA present. Increased ratios of indo-1 fluorescence indicate increased [Ca²⁺]ᵢ. Results represent those from at least four experiments.

FIG. 12B presents assays of tyrosine phosphorylation of proteins from purified splenic B cells of CD20^{-/-} and wild type mice. B cells (2×10⁷/sample) were incubated with anti-IgM antibody for the times shown and detergent lysed. Proteins were resolved by SDS-PAGE, transferred to nitrocellulose and immunoblotted with anti-phosphotyrosine (anti-PTyr) antibody. The blot was stripped and reprobed with anti-SHP-1 antibody as a control for equivalent protein loading. Western blots from two of three experiments are shown to demonstrate the range of results.

### DETAILED DESCRIPTION OF THE INVENTION

The present invention provides isolated nucleic acids encoding MS4A polypeptides (representative embodiments set forth as the odd-numbered SEQ ID NOs:1-37), isolated MS4A polypeptides (representative embodiments set forth as the even-numbered SEQ ID NOs:2-38), and uses thereof. The disclosed MS4A nucleic acids and polypeptides can be used according to methods of the present invention for drug discovery screens, for therapeutic treatment of atopic conditions, and for therapeutic regulation of [Ca²⁺]ₗ levels, among other uses.

I. Definitions

While the following terms are believed to be well understood by one of ordinary skill in the art, the following definitions are set forth to facilitate explanation of the invention. The entire contents of all publications mentioned herein, including the discussion of the background art presented above, are hereby fully incorporated by reference.

I.A. MS4A Nucleic Acids

The nucleic acid molecules provided by the present invention include the isolated nucleic acid molecules of any one of the odd-numbered SEQ ID NOs:1-37, sequences substantially similar to sequences of any one of the odd-numbered SEQ ID NOs:1-37, conservative variants thereof, subsequences and elongated sequences thereof, complementary DNA molecules, and corresponding RNA molecules. The present invention also encompasses genes, cDNAs, chimeric genes, and vectors comprising disclosed MS4A nucleic acid sequences.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides which have similar properties as the reference natural nucleic acid. Unless otherwise indicated, a particular nucleotide sequence also implicitly encompasses conservatively modified variants thereof (e.g. degenerate codon substitutions), complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a transgenic host cell.

The term “purified”, when applied to a nucleic acid, denotes that the nucleic acid is essentially free of other cellular components with which it is associated in the natural state. Preferably, a purified nucleic acid molecule is a homogeneous dry or aqueous solution. The term “purified” denotes that a nucleic acid or protein gives rise to essentially one band in an electrophoretic gel. Particularly, it means that the nucleic acid is at least about 50% pure, more preferably at least about 85% pure, and most preferably at least about 99% pure.

The term “substantially identical”, the context of two nucleotide or amino acid sequences, can also be defined as two or more sequences or subsequences that have at least 60%, preferably 80%, more preferably 90-95%, and most preferably at least 99% nucleotide or amino acid sequence identity, when compared and aligned for maximum correspondence, as measured using one of the following sequence comparison algorithms (described herein below under the heading Nucleotide and Amino Acid Sequence Comparisons) or by visual inspection. Preferably, the substantial identity exists in nucleotide sequences of at least 50 residues, more preferably in nucleotide sequence of at least about 100 residues, more preferably in nucleotide sequences of at least about 150 residues, and most preferably in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogenous population of nucleic acid molecules. A “target sequence” is synonymous with a “test sequence”.

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any of those set forth as the odd-numbered SEQ ID NOs:1-37. Such fragments can be readily prepared by, for example, directly synthesizing the fragment by chemical synthesis, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA). The phrase “binds substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.15 M NaCl at 65° C. An example of stringent wash conditions is 15 minutes in 0.2×SSC buffer at 65° C. (See. Sambrook et al. eds. (1989) Molecular Cloning: A Laboratory Manual, Cold Spring Harbor Laboratory Press, Cold Spring Harbor, N.Y. for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6× SSC at 40° C. For short probes (e.g., about 10 to 50 nucleotides), stringent conditions typically involve salt concentrations of less than about 1.0 M Na⁺ ion, typically about 0.01 to 1.0 M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the present invention: a probe nucleotide sequence preferably hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 1× SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.5× SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.1×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, are biologically functional equivalents, or are immunologically cross-reactive. These terms are defined further under the heading MS4A Polypeptides herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues (Batzer et al. (1991) Nucleic Acids Res 19:5081; Ohtsuka et al. (1985) J Biol Chem 260:2605-2608; Rossolini et al. (1994) Mol Cell Probes 8:91-98).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase), e.g., a polymerase which adds sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequence”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” generally refers to the cellular processes by which a biologically active polypeptide is produced from a DNA sequence.

The present invention also encompasses chimeric genes comprising the disclosed MS4A sequences. The term “chimeric gene”, as used herein, refers to a promoter region operably linked to a MS4A coding sequence, a nucleotide sequence producing an antisense RNA molecule, a RNA molecule having tertiary structure, such as a hairpin structure, or a double-stranded RNA molecule.

The term “operably linked”, as used herein, refers to a promoter region that is connected to a nucleotide sequence in such a way that the transcription of that nucleotide sequence is controlled and regulated by that promoter region. Techniques for operatively linking a promoter region to a nucleotide sequence are well known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native cis-regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “promoter region” defines a nucleotide sequence within a gene that is positioned 5′ to a coding sequence of a same gene and functions to direct transcription of the coding sequence. The promoter region includes a transcriptional start site and at least one cis-regulatory element. The present invention encompasses nucleic acid sequences that comprise a promoter region of a MS4A gene, or functional portion thereof.

The term “cis-acting regulatory sequence” or “cis-regulatory motif” or “response element”, as used herein, each refer to a nucleotide sequence that enables responsiveness to a regulatory transcription factor.

Responsiveness can encompass a decrease or an increase in transcriptional output and is mediated by binding of the transcription factor to the DNA molecule comprising the response element.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, and any other relevant protein that impacts gene transcription.

A “functional portion” of a promoter gene fragment is a nucleotide sequence within a promoter region that is required for normal gene transcription. To determine nucleotide sequences that are functional, the expression of a reporter gene is assayed when variably placed under the direction of a promoter region fragment.

Promoter region fragments can be conveniently made by enzymatic digestion of a larger fragment using restriction endonucleases or DNAse I. Preferably, a functional promoter region fragment comprises about 5000 nucleotides, more preferably 2000 nucleotides, more preferably about 1000 nucleotides. Even more preferably a functional promoter region fragment comprises about 500 nucleotides, even more preferably a functional promoter region fragment comprises about 100 nucleotides, and even more preferably a functional promoter region fragment comprises about 20 nucleotides.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operably linked to a transcriptional regulatory region can be found in Alam & Cook (1990) Anal Biochem 188:245-254 and PCT International Publication No. WO 97/47763. Preferred reporter genes for transcriptional analyses include the lacZ gene (See, e.g., Rose & Botstein (1983) Meth Enzymol 101:167-180), Green Fluorescent Protein (GFP) (Cubiff et al. (1995) Trends Biochem Sci 20:448-455), luciferase, or chloramphenicol acetyl transferase (CAT). Preferred reporter genes for methods to produce transgenic animals include but are not limited to antibiotic resistance genes, and more preferably the antibiotic resistance gene confers neomycin resistance. Any suitable reporter and detection method can be used, and it will be appreciated by one of skill in the art that no particular choice is essential to or a limitation of the present invention.

An amount of reporter gene can be assayed by any method for qualitatively or preferably, quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, preferably an increase by about 2-fold or greater relative to a control measurement, more preferably an increase by about 5-fold or greater, and most preferably an increase by about 10-fold or greater.

The present invention further includes vectors comprising the disclosed MS4A sequences, including plasmids, cosmids, and viral vectors. The term “vector”, as used herein refers to a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of a MS4A polypeptide, as described further herein below. Preferred vectors include but are not limited to pBluescript (Stratagene), pUC18, pBLCAT3 (Luckow & Schutz (1987) Nucleic Acids Res 15:5490), pLNTK (Gorman et al. (1996) Immunity 5:241-252), and pBAD/gIII (Stratagene). A preferred host cell is a mammalian cell; more preferably the cell is a Chinese hamster ovary cell, a HeLa cell, a baby hamster kidney cell, or a mouse cell; even more preferably the cell is a human cell.

Nucleic acids of the present invention can be cloned, synthesized, recombinantly altered, mutagenized, or combinations thereof. Standard recombinant DNA and molecular cloning techniques used to isolate nucleic acids are well known in the art. Exemplary, non-limiting methods are described by Sambrook et al., eds. (1989); by Silhavy et al. (1984) Experiments with Gene Fusions, Cold Spring Harbor Laboratory Press, Cold Spring Harbor, N.Y.; by Ausubel et al. (1992) Current Protocols in Molecular Biology, John Wylie and Sons, Inc., New York, N.Y.; and by Glover, ed. (1985) DNA Cloning: A Practical Approach, MRL Press, Ltd., Oxford, United Kingdom. Site-specific mutagenesis to create base pair changes, deletions, or small insertions are also well known in the art as exemplified by publications, see, e.g., Adelman et al., (1983) DNA 2:183; Sambrook et al. (1989).

Sequences detected by methods of the invention can be detected, subcloned, sequenced, and further evaluated by any measure well known in the art using any method usually applied to the detection of a specific DNA sequence including but not limited to dideoxy sequencing, PCR, oligomer restriction (Saiki et al. (1985) Bio/Technology 3:1008-1012), allele-specific oligonucleotide (ASO) probe analysis (Conner et al. (1983) Proc Natl Acad Sci USA 80:278), and oligonucleotide ligation assays (OLAs) (Landgren et. al. (1988) Science 241:1007). Molecular techniques for DNA analysis have been reviewed (Landgren et. al. (1988) Science 242:229-237).

I.B. MS4A Polypeptides

The polypeptides provided by the present invention include the isolated polypeptides set forth as the even-numbered SEQ ID NOs:2-38, polypeptides substantially similar to the even-numbered SEQ ID NOs:2-38, MS4A polypeptide fragments, fusion proteins comprising MS4A amino acid sequences, biologically functional analogs, and polypeptides that cross-react with an antibody that specifically recognizes a MS4A polypeptide.

The term “isolated”, as used in the context of a polypeptide, indicates that the polypeptide exists apart from its native environment and is not a product of nature. An isolated polypeptide can exist in a purified form or can exist in a non-native environment such as, for example, in a transgenic host cell.

The term “purified”, when applied to a polypeptide, denotes that the polypeptide is essentially free of other cellular components with which it is associated in the natural state. Preferably, a polypeptide is a homogeneous solid or aqueous solution. Purity and homogeneity are typically determined using analytical chemistry techniques such as polyacrylamide gel electrophoresis or high performance liquid chromatography. A polypeptide which is the predominant species present in a preparation is substantially purified. The term “purified” denotes that a polypeptide gives rise to essentially one band in an electrophoretic gel. Particularly, it means that the polypeptide is at least about 50% pure, more preferably at least about 85% pure, and most preferably at least about 99% pure.

The term “substantially identical” in the context of two or more polypeptides sequences is measured by (a) polypeptide sequences having about 35%, or 45%, or preferably from 45-55%, or more preferably 55-65%, or most preferably 65% or greater amino acids which are identical or functionally equivalent. Percent “identity” and methods for determining identity are defined herein below under the heading Nucleotide and Amino Acid Sequence Comparisons.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Henikoff et al. (2000) Electrophoresis 21(9):1700-1706; Huang et al. (2000) Pac Symp Biocomput 230-241; Saqi et al. (1999) Bioinformatics 15(6):521-522; and Barton (1998) Acta Crystallogr D Biol Crystallogr 54:1139-1146.

The term “functionally equivalent” in the context of amino acid sequences is well known in the art and is based on the relative similarity of the amino acid side-chain substituents. See Henikoff & Henikoff (2000) Adv Protein Chem 54:73-97. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (4.5).

The importance of the hydropathic amino acid index in conferring interactive biological function on a protein is generally understood in the art (Kyte et al. (1982) J Mol Biol 157:105.). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The present invention also encompasses MS4A polypeptide fragments or functional portions of a MS4A polypeptide. Such functional portion need not comprise all or substantially all of the amino acid sequence of a native MS4A gene product. The term “functional” includes any biological activity or feature of MS4A, including immunogenicity.

The present invention also includes longer sequences of a MS4A polypeptide, or portion thereof. For example, one or more amino acids can be added to the N-terminus or C-terminus of a MS4A polypeptide. Fusion proteins comprising MS4A polypeptide sequences are also provided within the scope of the present invention. Methods of preparing such proteins are known in the art.

The present invention also encompasses functional analogs of a MS4A polypeptide. Functional analogs share at least one biological function with a MS4A polypeptide. An exemplary function is immunogenicity. In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In one embodiment, changes can be introduced to improve the antigenicity of the protein. In another embodiment, a MS4A polypeptide sequence is varied so as to assess the activity of a mutant MS4A polypeptide.

The present invention also encompasses recombinant production of the disclosed MS4A polypeptides. Briefly, a nucleic acid sequence encoding a MS4A polypeptide, or portion thereof, is cloned into a expression cassette, the cassette is introduced into a host organism, where it is recombinantly produced.

The term “expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest can be chimeric. The expression cassette can also be one which is naturally occurring but has been obtained in a recombinant form useful for heterologous expression.

The expression of the nucleotide sequence in the expression cassette can be under the control of a constitutive promoter or an inducible promoter which initiates transcription only when the host cell is exposed to some particular external stimulus. Exemplary promoters include Simian virus 40 early promoter, a long terminal repeat promoter from retrovirus, an action promoter, a heat shock promoter, and a metallothien protein. In the case of a multicellular organism, the promoter and promoter region can direct expression to a particular tissue or organ or stage of development. Exemplary tissue-specific promoter regions include a MS4A promoter, described herein. Suitable expression vectors which can be used include, but are not limited to, the following vectors or their derivatives: human or animal viruses such as vaccinia virus or adenovirus, yeast vectors, bacteriophage vectors (e.g., lambda phage), and plasmid and cosmids DNA vectors.

The term “host cell”, as used herein, refers to a cell into which a heterologous nucleic acid molecule has been introduced. Transformed cells, tissues, or organisms are understood to encompass not only the end product of a transformation process, but also transgenic progeny thereof.

A host cell strain can be chosen which modulates the expression of the inserted sequences, or modifies and processes the gene product in the specific fashion desired. For example, different host cells have characteristic and specific mechanisms for the translational and post-transactional processing and modification (e.g., glycosylation, phosphorylation of proteins). Appropriate cell lines or host systems can be chosen to ensure the desired modification and processing of the foreign protein expressed. Expression in a bacterial system can be used to produce a non-glycosylated core protein product. Expression in yeast will produce a glycosylated product. Expression in animal cells can be used to ensure “native” glycosylation of a heterologous protein.

Expression constructs are transfected into a host cell by any standard method, including electroporation, calcium phosphate precipitation, DEAE-Dextran transfection, liposome-mediated transfection, and infection using a retrovirus. The MS4A-encoding nucleotide sequence carried in the expression construct can be stably integrated into the genome of the host or it can be present as an extrachromosomal molecule.

Isolated polypeptides and recombinantly produced polypeptides can be purified and characterized using a variety of standard techniques that are well known to the skilled artisan. See e.g. Ausubel et al. (1992), Bodanszky, et al. (1976) Peptide Synthesis, John Wiley and Sons, Second Edition, New York, N.Y. and Zimmer et al. (1993) Peptides, pp. 393-394, ESCOM Science Publishers, B. V.

I.C. Nucleotide and Amino Acid Sequence Comparisons

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain at least some of biological activity of the natural gene, gene product, or sequence. Such sequences include “mutant” sequences, or sequences wherein the biological activity is altered to some degree but retains at least some of the original biological activity. The term “naturally occurring”, as used herein, is used to describe a composition that can be found in nature as distinct from being artificially produced by man. For example, a protein or nucleotide sequence present in an organism, which can be isolated from a source in nature and which has not been intentionally modified by man in the laboratory, is naturally occurring.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482, by the homology alignment algorithm of Needleman & Wunsch (1970) J Mol Biol 48:443, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel et al., 1992.

A preferred algorithm for determining percent sequence identity and sequence similarity is the BLAST algorithm, which is described in Altschul et al. (1990) J Mol Biol 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1989) Proc Natl Acad Sci USA 89:10915.

In addition to calculating percent sequence identity, the BLAST algorithm also performs a statistical analysis of the similarity between two sequences. See e.g., Karlin and Altschul (1993) Proc Natl Acad Sci USA 90:5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

I.D. Antibodies

The present invention also provides an antibody that specifically binds a MS4A polypeptide. The term “antibody” indicates an immunoglobulin protein, or functional portion thereof, including a polyclonal antibody, a monoclonal antibody, a chimeric antibody, a single chain antibody, Fab fragments, and an Fab expression library. “Functional portion” refers to the part of the protein that binds a molecule of interest. In a preferred embodiment, an antibody of the invention is a monoclonal antibody. Techniques for preparing and characterizing antibodies are well known in the art (See, e.g., Harlow & Lane (1988) Antibodies: A Laboratory Manual, Cold Spring Harbor Laboratory Press, Cold Spring Harbor, N.Y.). A monoclonal antibody of the present invention can be readily prepared through use of well-known techniques such as the hybridoma techniques exemplified in U.S. Pat. No. 4,196,265 and the phage-displayed techniques disclosed in U.S. Pat. No. 5,260,203.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected for its specificity for a particular protein. For example, antibodies raised to a protein with an amino acid sequence encoded by any of the nucleic acid sequences of the invention can be selected to obtain antibodies specifically immunoreactive with that protein and not with unrelated proteins.

The use of a molecular cloning approach to generate antibodies, particularly monoclonal antibodies, and more particularly single chain monoclonal antibodies, are also provided. The production of single chain antibodies has been described in the art. See, e.g., U.S. Pat. No. 5,260,203. For this approach, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody of the present invention, or a “derivative” of an antibody of the present invention, pertains to a single polypeptide chain binding molecule which has binding specificity and affinity substantially similar to the binding specificity and affinity of the light and heavy chain aggregate variable region of an antibody described herein.

The term “immunochemical reaction”, as used herein, refers to any of a variety of immunoassay formats used to detect antibodies specifically bound to a particular protein, including but not limited to competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, and immunoelectrophoresis assays, etc. See Harlow & Lane (1988) for a description of immunoassay formats and conditions.

I.E. Protein Binding Assays

The term “binding” refers to an affinity between two molecules, for example, a ligand and a receptor. As used herein, “binding” means a preferential binding of one molecule for another in a mixture of molecules. The binding of the molecules can be considered specific if the binding affinity is about 1×10⁴ M⁻¹ to about 1×10⁶ M⁻¹ or greater. Binding of two molecules also encompasses a quality or state of mutual action such that an activity of one protein or compound on another protein is inhibitory (in the case of an antagonist) or enhancing (in the case of an agonist). Exemplary protein binding assays include but are not limited to Fluorescence Correlation Spectroscopy (FCS), Surface-Enhanced Laser Desorption/Ionization time-of-flight mass spectrometry (SELDI-TOF), and Biacore, each described further herein below.

Fluorescence Correlation Spectroscopy (FCS) measures the average diffusion rate of a fluorescent molecule within a small sample volume (Madge et al. (1972) Phys Rev Left 29:705-708; Maiti et al. (1997) Proc Natl Acad Sci USA, 94:11753-11757). The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N-terminus or C-terminus. The expression takes place in E. coli, yeast or mammalian cells. The protein is purified using chromatographic methods. For example, the poly-histidine tag can be used to bind the expressed protein to a metal chelate column such as Ni²⁺ chelated on iminodiacetic acid agarose. The protein is then labeled with a fluorescent tag such as carboxytetramethylrhodamine or BODIPY™ (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was developed by Hutchens & Yip (1993) Rapid Commun Mass Spectrom 7:576-580). When coupled to a time-of-flight mass spectrometer (TOF), SELDI provides a means to rapidly analyze molecules retained on a chip. It can be applied to ligand-protein interaction analysis by covalently binding the target protein on the chip and analyzing by MS the small molecules that bind to this protein (Worrall et al. (1998) Anal Biochem 70:750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind the target are identified by the stringency of the wash needed to elute them.

Biacore relies on changes in the refractive index at the surface layer upon binding of a ligand to a protein immobilized on the layer. In this system, a collection of small ligands is injected sequentially in a 2-5 microliter cell, wherein the protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al. (1983) Sensors Actuators 4:299-304; Malmquist (1993) Nature 361:186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction.

I.F. Transgenic Animals

It is also within the scope of the present invention to prepare a transgenic animal to mutagenize the MS4A locus or to express a transgene comprising nucleic acid sequences of the present invention. The term “transgenic animal”, indicates an animal comprising a germline insertion of a heterologous nucleic acid. Transgenic animals of the present invention are understood to encompass not only the end product of a transformation method, but also transgenic progeny thereof.

The term “transgene”, as used herein indicates a heterologous nucleic acid molecule that has been transformed into a host cell. For intended use in the creation of a transgenic animal, the transgene includes genomic sequences of the host organism at a selected locus or site of transgene integration to mediate a homologous recombination event. A transgene further comprises nucleic acid sequences of interest, for example a targeted modification of the gene residing within the locus, a reporter gene, or a expression cassette, each defined herein above.

Transgene integration can be used to create gene mutations, including “knock-out”, “knock-in”, or a “knock-down” mutations. Representative approaches are disclosed in the Examples presented below. The term “knock-out” refers to a homologous recombination event that renders a gene inactive. Gene knock-out is generally accomplished by integration of the transgene at a chromosomal loci, thereby interrupting a gene residing at that loci. The term “knock-in” refers to in vivo replacement at a targeted locus. Knock-in mutations can modify a gene sequence to create a loss-of-function or gain-of-function mutation. The term “gene knock-down” refers to a homologous recombination event wherein the transgene partially eliminates gene function. A knock-down animal can be created by transgenic expression of an antisense molecule, wherein a transgene comprising the antisense sequence and a relevant promoter are integrated into the genome at a non-essential loci. Expression of the antisense or ribozyme molecule disrupts the corresponding gene function, although this disruption is generally incomplete (Luyckx et al. (1999) Proc Natl Acad Sci U S A 96(21):12174-12179).

Conditional mutation can be accomplished using transgenic methods in combination with the Cre-recombinase system in mice. Briefly, in one instance, a transgenic mouse is derived that expresses Cre-recombinase under the direction of an inducible promoter. A second transgenic mouse bears a mutation of a gene of interest as well as a lox-P-flanked endogenous gene sequence. Such transgenic mice are mated, the resulting progeny having both the Cre-recombinase and lox-P-flanked transgenes. Induction of Cre recombinase catalyzes excision of the lox-P-flanked transgene, thereby excising a portion of the endogenous gene sequence and revealing the mutated sequence. Conditional knockout can be varied according to the temporal and spatial features of Cre recombinase expression, inherent in the selection of a promoter to drive Cre recombinase. See Postic et al. (1999) J Biol Chem 275(1):305-315; and Sauer (1998) Methods 14(4):381-392.

Transgenes can also be used for heterologous expression in a host organism without generating phenotypically apparent mutations. By this method, nucleotide sequences of interest are introduced into the genome at a nonessential loci, whereby insertion alone does not disrupt an essential gene function. Optionally, expression of the transgene can generate a gain-of-function or ectopic function phenotype.

Techniques for the preparation of transgenic animals are known in the art. Exemplary techniques are described in U.S. Pat. No. 5,489,742 (transgenic rats); U.S. Pat. Nos. 4,736,866, 5,550,316, 5,614,396, 5,625,125 and 5,648,061 (transgenic mice); U.S. Pat. No. 5,573,933 (transgenic pigs); U.S. Pat. No. 5,162,215 (transgenic avian species) and U.S. Pat. No. 5,741,957 (transgenic bovine species). Briefly, nucleotide sequences of interest are cloned into a vector, and the construct is transformed into a germ cell. In the germ cell, a chromosomal rearrangement event takes place wherein the nucleic acid sequences of interest are integrated into the genome of the germ cell by homologous recombination. Fertilization and propagation of the transformed germ cell results in a transgenic animal. Homozygosity of the mutation is accomplished by intercrossing.

I.G. Therapeutic Methods

The present invention further provides methods for discovering substances that can be used as pharmaceutical compositions. The term “pharmaceutical composition” or “drug” as used herein, each refer to any substance having a biological activity. Substances discovered by methods of the present invention include but are not limited to polypeptide, proteins, peptides, chemical compounds, and antibodies.

A composition of the present invention is typically formulated using acceptable vehicles, adjuvants, and carriers as desired.

Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectable compositions.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic diluent or solvent, for example 1,3-butanediol.

A vector can be used as a carrier, for example an adenovirus vector, can be used for gene therapy methods. The vector is purified to sufficiently render it essentially free of undesirable contaminants, such as defective interfering adenovirus particles or endotoxins and other pyrogens such that it does not cause any untoward reactions in the individual receiving the vector construct. A preferred means of purifying the vector involves the use of buoyant density gradients, such as cesium chloride gradient centrifugation.

A transfected cell can also serve as a carrier. By way of example a liver cell can be removed from an organism, transfected with a nucleic acid sequence of the present invention using methods set forth above and then the transfected cell returned to the organism (e.g. injected intra-vascularly).

Monoclonal antibodies or polypeptides of the invention can be administered parenterally by injection or by gradual infusion over time. Although the tissue to be treated can typically be accessed in the body by systemic administration and therefore most often treated by intravenous administration of therapeutic compositions, other tissues and delivery means are provided where there is a likelihood that the tissue targeted contains the target molecule and are known to those of skill in the art.

Representative antibodies for use in the present invention are intact immunoglobulin molecules, substantially intact immunoglobulin molecules, single chain immunoglobulins or antibodies, those portions of an immunoglobulin molecule that contain the paratope, including antibody fragments. It is within the scope of the present invention that a monovalent modulator can optionally be used.

Methods of preparing “humanized” antibodies are generally well known in the art, and can readily be applied to the antibodies of the present invention. Humanized monoclonal antibodies offer particular advantages over monoclonal antibodies derived from other mammals, particularly insofar as they can be used therapeutically in humans. Specifically, humanized antibodies are not cleared from the circulation as rapidly as “foreign” antigens, and do not activate the immune system in the same manner as foreign antigens and foreign antibodies.

With respect to the therapeutic methods of the present invention, a preferred subject is a vertebrate subject. A preferred vertebrate is warm-blooded; a preferred warm-blooded vertebrate is a mammal. A preferred mammal is a mouse or, most preferably, a human. As used herein and in the claims, the term “patient” includes both human and animal patients. Thus, veterinary therapeutic uses are provided in accordance with the present invention.

Also provided is the treatment of mammals such as humans, as well as those mammals of importance due to being endangered, such as Siberian tigers; of economical importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, provided is the treatment of livestock, including, but not limited to, domesticated swine, ruminants, ungulates, horses, poultry, and the like.

As used herein, the term “experimental subject” refers to any subject or sample in which the desired measurement is unknown. The term “control subject” refers to any subject or sample in which a desired measure is unknown.

As used herein, an “effective” dose refers to a dose(s) administered to an individual patient sufficient to cause a change in MS4A activity. One of ordinary skill in the art can tailor the dosages to an individual patient, taking into account the particular formulation and method of administration to be used with the composition as well as patient height, weight, severity of symptoms, and stage of the biological condition to be treated. Such adjustments or variations, as well as evaluation of when and how to make such adjustments or variations, are well known to those of ordinary skill in the art of medicine.

A therapeutically effective amount can comprise a range of amounts. One skilled in the art can readily assess the potency and efficacy of a MS4A modulator of this invention and adjust the therapeutic regimen accordingly. A modulator of MS4A biological activity can be evaluated by a variety of means including the use of a responsive reporter gene, interaction of MS4A polypeptides with a monoclonal antibody, analysis of cell subpopulations, and measurement of [Ca²⁺]ᵢ levels, each technique described herein.

Additional formulation and dose techniques have been described in the art, see for example, those described in U.S. Pat. Nos. 5,326,902 and 5,234,933, and International Publication No. WO 93/25521.

For the purposes described above, the identified substances can normally be administered systemically, parenterally, or orally. The term “parenteral” as used herein includes intravenous, intramuscular, intra-arterial injection, or infusion techniques. Other compositions for administration include liquids for external use, and endermic liniments (ointment, etc.), suppositories, and pessaries which comprise one or more of the active substance(s) and can be prepared by known methods.

II. CD20 Gene Family Members

II.A. Identification of CD20 Gene Family Members

The present invention provides MS4A nucleic acid and polypeptide sequences. Preferably, a MS4A gene comprises the sequence set forth as any one of the odd-numbered SEQ ID NOs:1-37, a nucleic acid molecule that is substantially similar to any one of the odd-numbered SEQ ID NOs:1-37, or a nucleic acid molecule comprising a 20 base pair nucleotide sequence that is identical to a contiguous 20 base pair sequence of any one of the odd-numbered SEQ ID NOs:1-37.

To identify new CD20 gene family members, the human and mouse CD20 amino acid sequences (Tedder et al., 1988a; Tedder et al., 1988b) were used to search the translated GenBank databases, including expressed sequence tags, using the BLAST program (Altschul et al., 1997).

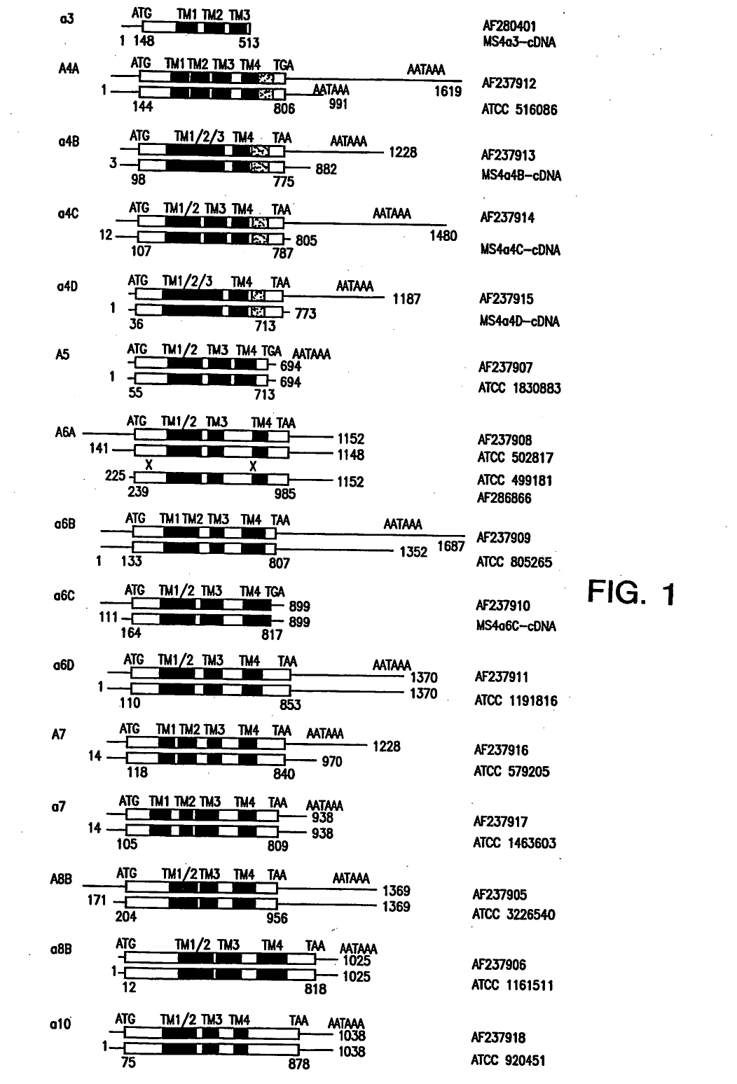

Among 337 homologous sequences identified, at least 17 novel genes expressed by mouse, human, and pig had predicted amino acid sequences homologous to CD20. Complete coding regions were predicted using overlapping nucleotide sequences obtained from sequenced ESTs and cDNAs that corresponded to unique, near full-length transcripts in humans and mice (FIG. 1). All nucleotide sequences were verified by sequencing multiple near full-length cDNAs isolated by applicants and 40 cDNAs obtained from the ATCC (American Tissue Culture Collection, Bethesda, Md., USA). In addition, a pig cDNA and its human counterpart homologous to CD20 were identified as GenBank submissions AJ236932.1 and AK000224, respectively. In total, unique cDNA clones were identified that encode at least 16 distinct full-length CD20-like proteins.

Complete cDNA sequences encoding the human and mouse MS4A family members (MS4A1, -A2, -A3, -A4A, -A5, -A6A, -A7, -A8B and -A12) were also used to search the GenBank human genomic database (htgs; http://www.ncbi.nlm.nih.gov/blast/) using the BLAST program (Altschul et al., 1997), as further described in Example 2. Two-hundred-twenty different contigs or distinct genomic DNA sequences were identified in the database of unfinished human genomic sequences that were either identical or similar to MS4A family members. These sequences were predominantly derived from sixteen partially sequenced bacterial artificial chromosomes (BACs) that spanned 400-500 kb of human chromosome 11q12 (Table 1). Based on known cDNA sequences of MS4A family members, we were able to order and arrange these genomic sequences into overlapping continuous DNA segments. Since many of the contigs identified were overlapping, it was thereby possible to assemble long DNA sequences that encoded entire MS4A genes or portions of MS4A genes. Gaps between exon encoding DNA sequences were filled in many cases by additional sequence homology searches using DNA sequences found at the ends of gaps. When sequence differences were observed between different overlapping DNA fragments, consensus sequences were used or PCR primers were generated, that portion of genomic DNA was then amplified and sequenced to resolve ambiguous sequences.

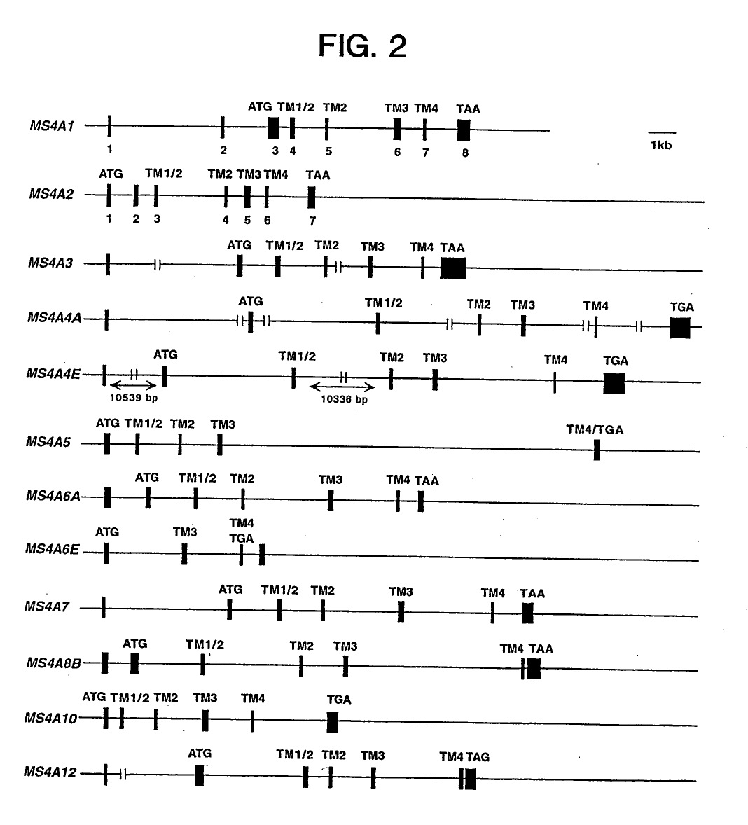

BLAST analysis of the htgs phase 1 or phase 2 human genomic DNA sequences encoding MS4A cDNAs and the assembled and annotated human genomic sequence thereof, as disclosed herein, revealed the presence of each known human MS4A family member. In addition, three putative genes encoding unique MS4A family members were identified that localized to the q12-13.1 region of human chromosome 11. Complete coding regions were predicted using overlapping nucleotide sequences obtained from sequenced ESTs and cDNAs and by comparison of gene structure, described further herein below (FIG. 2).

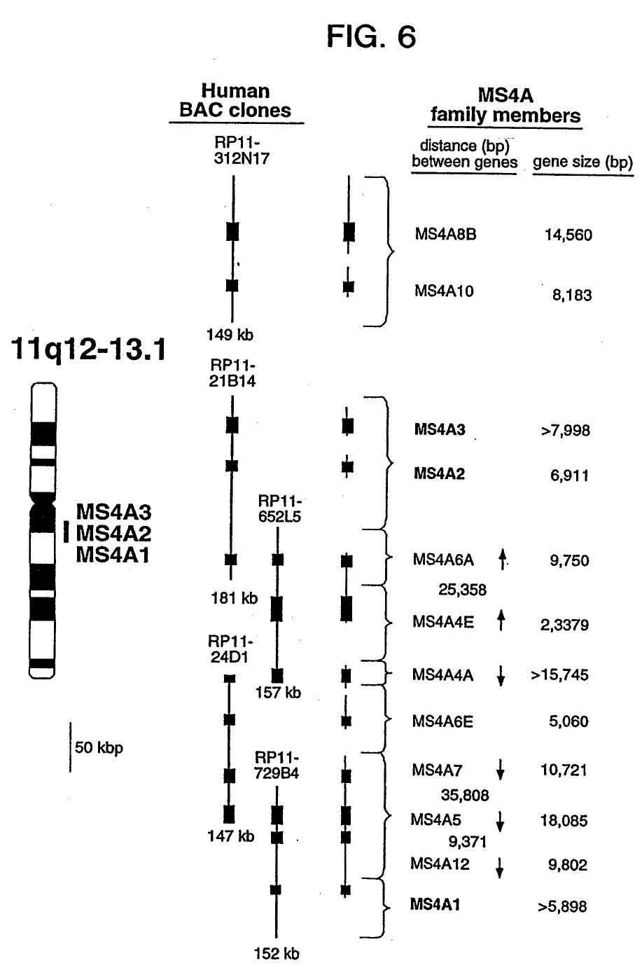

By identifying sequences that correlated with different MS4A genes in each BAC (Table 1), and by the assembly of minimal genomic DNA lengths that could encode each MS4A gene (FIG. 2), we used the overlapping BACs to identify the order of the MS4A genes on chromosome 11q12 (FIG. 6). This analysis also allowed us to determine the direction of gene transcription for most MS4A genes. Furthermore, the MS4A cDNA sequences, disclosed herein, were used to assemble genomic clones set forth as SEQ ID NOs:73-81. In some cases, multiple MS4A genes could be aligned within a continuous genomic sequence. For example, the genomic sequence set forth as SEQ ID NO:77 comprises both the MS4A4E and MS4A6A genes. Similarly, the genomic region set forth as SEQ ID NO:79 comprises three MS4A genes: MS4A7, MS4A5, and MS4A12.

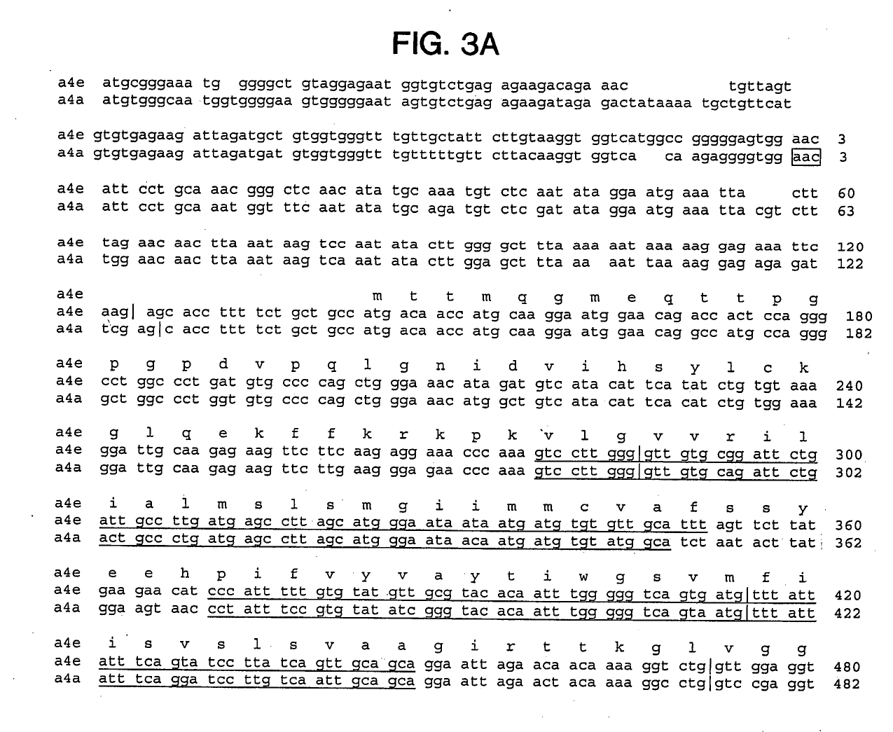

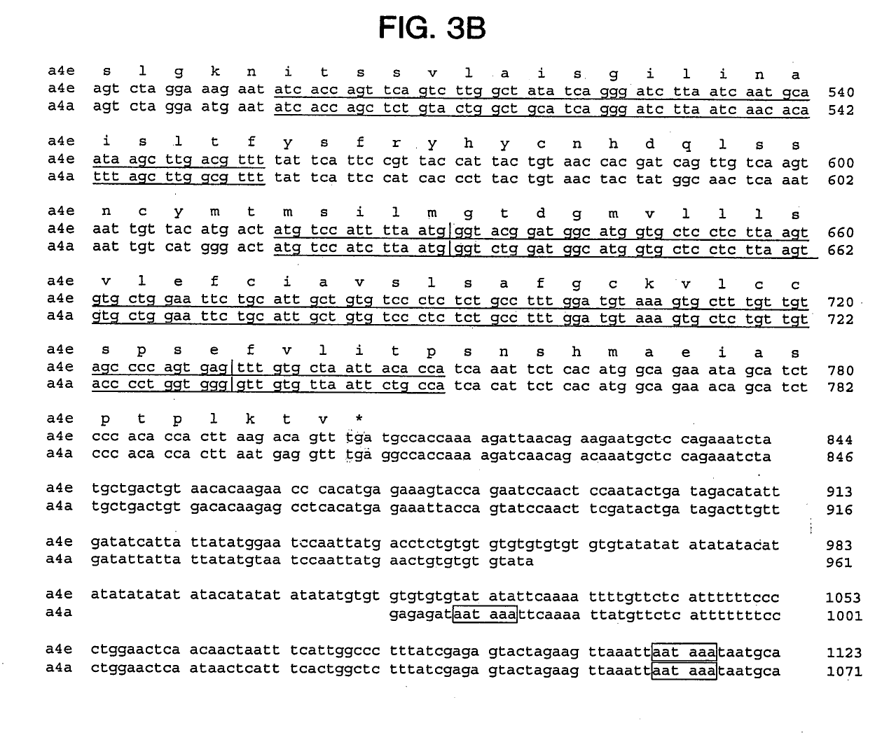

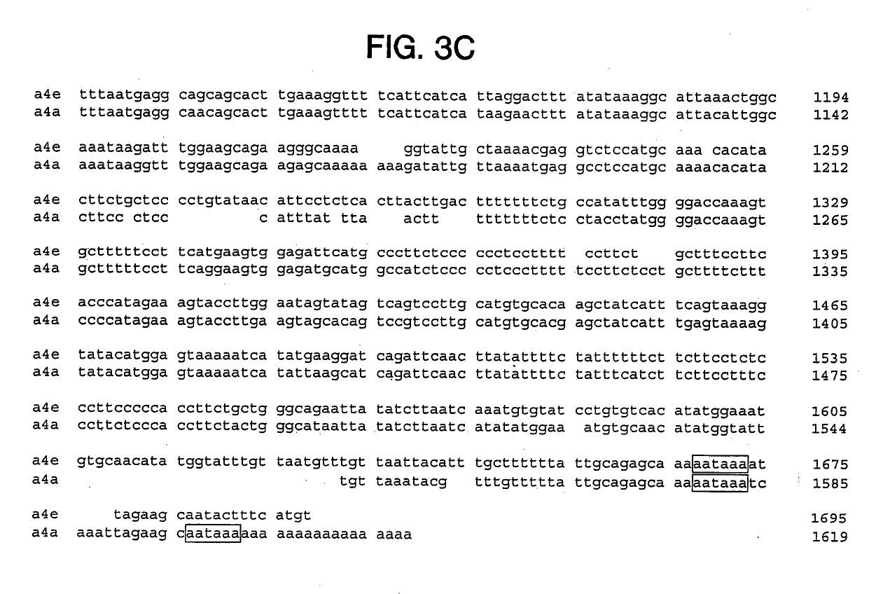

The MS4A4E gene encodes 660 bp of translated sequence (FIG. 3), contained within at least seven exons (FIG. 2). Exons were identified based on their sequence similarities with MS4A4A sequences and the identification of canonical splice-donor and -acceptor sites (Aebi & Weissmann, 1987). The MS4A4E gene sequence was at least 23,379 base pairs in length, if counted from the putative translation initiation ATG site until the TGA translation termination stop site (FIG. 2). An exon encoding the putative 5′ untranslated region of MS4A4E, was highly homologous with the corresponding sequence in MS4A4A cDNAs (disclosed herein). This sequence homology extended for >7 kbp upstream from this putative exon and also included upstream repetitive Alu elements. Representative upstream homologous sequences are shown in FIG. 3. Similar sequence homologies were identified in the 3′ untranslated regions of MS4A4E and MS4A4A, which extended beyond the poly-A attachment signal sequences (FIG. 3). Based on the sequence similarities in translated and untranslated exons, it appears that the MS4A4E and MS4A4A genes resulted from a recent gene duplication event.

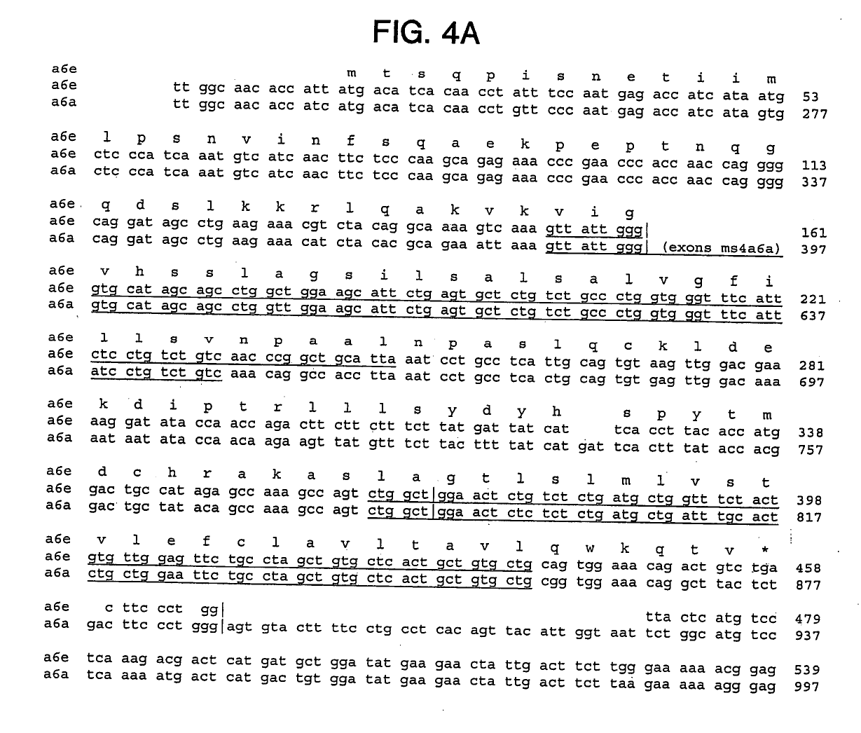

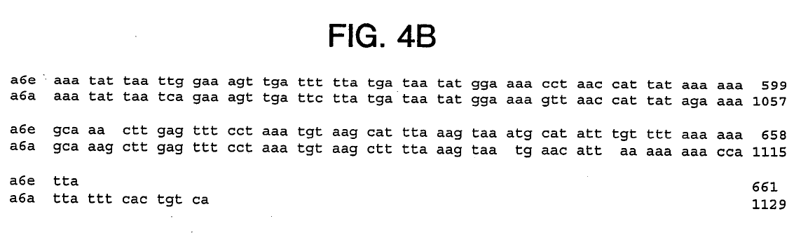

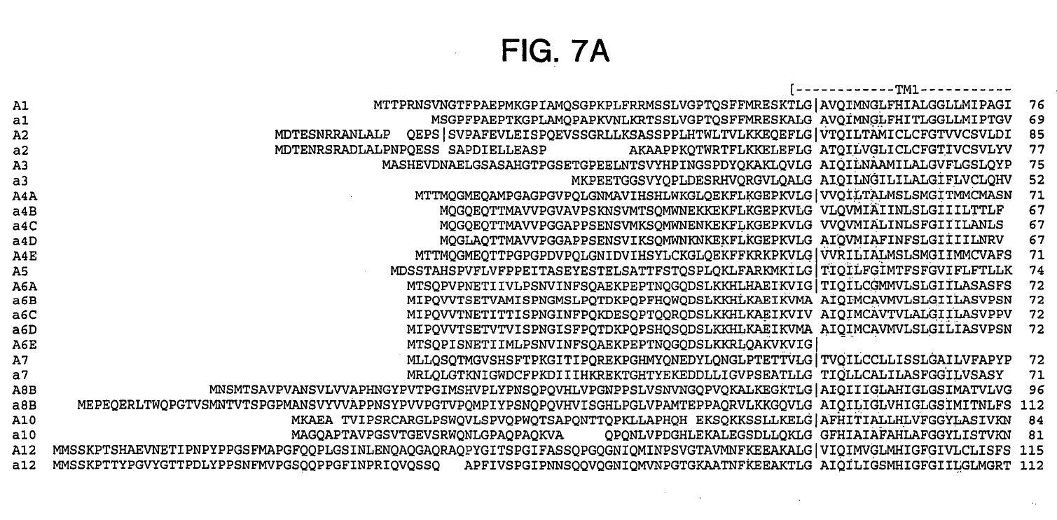

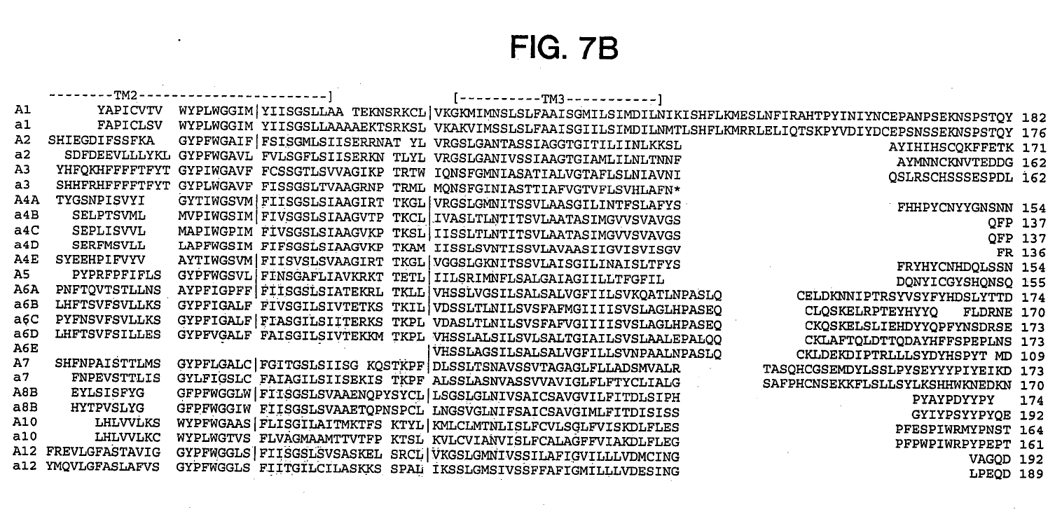

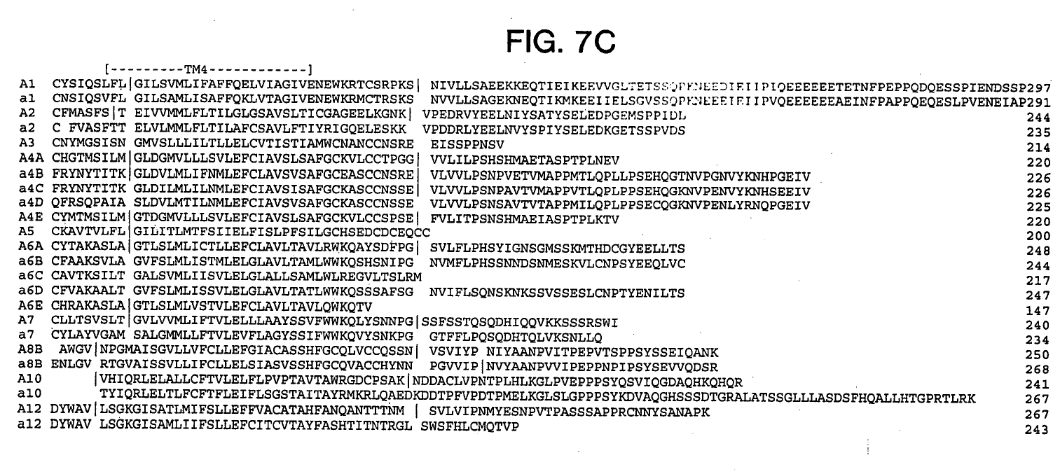

The MS4A6E gene encodes 441 bp of translated sequence (FIG. 4), contained within at least four exons (FIG. 2). Exons were identified based on their sequence similarities with MS4A6A cDNA sequences and the identification of canonical splice-donor and -acceptor sites (Aebi & Weissmann, 1987). In addition, the predicted gene sequences matched those found in three cDNA clones that were sequenced (ATCC Nos. 3704466, 1852248 and 3557769). The MS4A6E gene was at least 5,060 bp in length, if counted from the putative translation initiation ATG site until the TGA translation termination codon (FIG. 2). The MS4A6E gene lacks exons that encode the first two membrane spanning domains present in most MS4A family proteins (FIGS. 2 and 7). An exon homologous with the 5′ untranslated region of MS4A6A cDNAs was not identified within 7,629 bp of sequence upstream of the exon encoding the translation initiation site of MS4A6E. However, there was a canonical 3′ splice region upstream of the ATG initiation codon located at identical positions in the MS4A6E and MS4A6A genes. Similar sequence homologies were identified in the 3′ untranslated regions of MS4A6E and MS4A6A that extend beyond the sequence shown in FIG. 4. Based on the sequence similarities in translated and untranslated exons, it appears that the MS4A6E and MS4A6A genes represent a recent gene duplication event, although several exons encoding translated sequence were lost in the MS4A6E gene (FIG. 2).

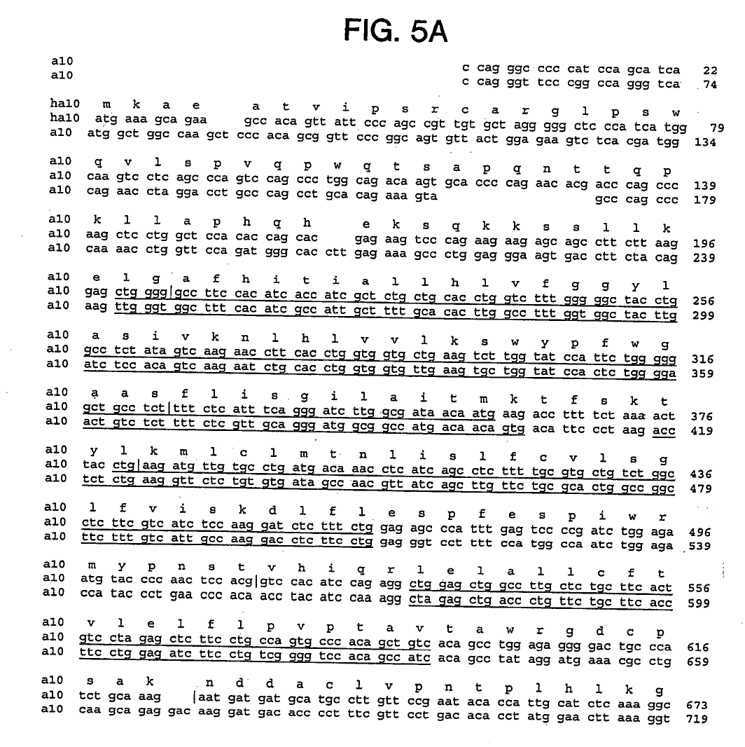

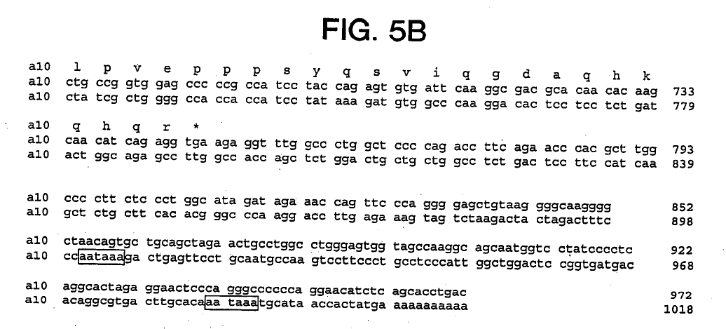

The MS4A10 gene encodes 726 bp of translated sequence (FIG. 5), contained within at least six exons (FIG. 2). Exons were identified based on their sequence similarities with mouse MS4A10 cDNA sequences and the identification of canonical splice-donor and -acceptor sites (Aebi & Weissmann, 1987). The MS4A10 gene was at least 8,183 bp in length if counted from the putative translation initiation ATG site until the TGA translation termination stop site (FIG. 2). An exon homologous with the 5′ untranslated region of mouse MS4A10 cDNAs was not identified within 8,829 bp of sequence upstream of the exon encoding the translation initiation site of MS4A10. However, there was a canonical 3′ splice region upstream of the ATG initiation codon located at identical positions in the MS4A10 and MS4A10 genes. Modest sequence homologies were identified in the 3′ untranslated regions of MS4A10 and MS4A10 (FIG. 5).

TABLE 1
Human BACs Containing MS4A Genes

| BAC | Accession No.^{a} | Chromosome | MS4A gene^{b} |
|---|---|---|---|
| RP11-206B10 | AC009703 | 15 | A4A, A4E, A6A |
| RP11-21B14 | AC013807 | unknown | A6A, A2, A3 |
| RP11-24D1 | AC015840 | unknown | A4A, A5, A6E, A7 |
| RP11-652L5 | AC018966 | 11 | A4A, A4E, A6A |
| RP11-448N3 | AC024066 | 11 | A8B |
| RP11-312N17 | AC027599 | 11 | A8B, A10 |
| RP11-196E16 | AC027787 | 15 | A5, A1 |
| CMB9-79B2 | AP000748 | 11q23 | A10 |
| RP11-804A23 | AP000777 | 11 | A10 |
| RP11-736I10 | AP000790 | 11q12 | A3 |
| RP11-804B24 | AP000934 | 11 | A10 |
| RP11-729B4 | AP001034 | 11q12 | A5, A12, A1 |
| CMB9-2M23 | AP001181 | 11q12 | A2, A3 |
| CMB9-100I1 | AP001257 | 11q12 | A6A, A4E |
| CMB9-49F18 | AP001259 | 11 | A8B |
| RP11-68H20 | AP001986 | 11q | A10 |

^{a}GenBank Accession number for the indicated BAC.
^{b}indicates the MS4A gene sequences that mapped to each BAC.

II.B. MS4A Nomenclature

In collaboration with the Human Gene Nomenclature Committee (www.gene.ucl.ac.uk/nomenclature/), this gene family was designated as the MS4A family (Membrane Spanning 4-domain family, subfamily A). The MS4 designation is to accommodate the future identification of genes encoding proteins with a similar structure, yet with unresolved functions. Subfamily A will designate the CD20 family. Using this nomenclature, the CD20 gene was designated as MS4A1, FcεRIβ as MS4A2, and HTm4 as MS4A3. Among the 16 novel genes identified, 8 human genes were named MS4A4A, MS4A4E, MS4A5, MS4A6A, MS4A6E, MS4A7, MS4A8B, and MS4A12. A ninth gene encoded a protein homologous with the single member of the mouse MS4A10 subfamily. This gene was tentatively designated as MS4A10. The remaining genes were of mouse or pig origin and were therefore labeled as MS4a3-MS4a12 based on the nomenclature of homologous genes corresponding to human counterparts. Distinct mouse genes that encoded proteins with highly homologous sequences were designated as MS4a4B, MS4a4C, MS4a4D, and as MS4a6B, MS4a6C, and MS4a6D to signify close homology.

II.C. MS4A Gene Chromosome Locations

Chromosome locations for the human MS4A4A, MS4A6A, MS4A7, and MS4A8B genes were identified in two distinct homology searches. Regions of human MS4A4A (bp 1286-1588), MS4A6A (bp 682-1106), MS4A7 (bp 502-941), MS4A7 (bp 1015-1177), and MS4A8B (bp 1007-1350), were 98%, 98%, 97%, 99% and 97% identical with human STS genomic sequence tag sites, WI-11578, SHGC-36634, WI-12101, WIAF-3856, and WI-14145, respectively (http://www.ncbi.nim.nih.gov/blast). These genomic sequence tag sites are located on human chromosome 11 at Genomic Database locus D11S1357-D11S913, which maps to 11q12-13 (http://www.ncbi.nlm.nih.gov/genemap). These mapping results were confirmed using the UniGene collection at the National Center for Biotechnology Information (http://www.ncbi.nlm.nih.gov/Genemap98/) for expressed sequence tags identical to human MS4A4A, MS4A6A, MS4A7, MS4A8B sequences. By this analysis, at least 7 of the 9 currently identified human MS4A genes are clustered.

The organization of the 12 MS4A genes on human chromosome 11 was determined by identifying sequenced human genomic DNA fragments (contigs of different lengths) from 15 BAC clones (Table 1). Contiguous DNA segments for each BAC were constructed based on human MS4A exon and cDNA sequences, and overlapping contigs. Although some gaps were present in MS4A gene introns (FIG. 2) or between MS4A genes, the relative position of each gene on chromosome 11q12-13.1 was determined (FIG. 6). MS4A1 was located in a telemetric region of 11q12-13.1 compared with MS4A2 and MS4A3. Seven MS4A genes were located in between MS4A1 and MS4A2. Two other MS4A genes, MS4A8B and MS4A10 were centromeric to MS4A2 and MS4A3, although the distance between these genes was not determined. Interestingly, MS4A6A, MS4A4E, MS4A4A and MS4A6E were arranged linearly suggesting that these genes might have arisen through the duplication of a single genomic element. It is envisioned that this genetic locus extends further and contains additional MS4A genes.

II.A. MS4A Gene Structure

Complete coding region sequences were verified for each deduced protein, except for the MS4a3 cDNA that was not full-length (FIG. 1). Proposed ATG translation initiation codons were based on the translation initiation consensus sequence, ANNATG (Kozak (1986) Cell 44:283-292), and the existence of in-frame upstream translation stop codons in most cases. Whether the first or second ATG codon in mouse MS4a8B was used for translation initiation was unknown although the second ATG was identical with the start codon of human MS4A8B (FIG. 7).

Poly(A) attachment signal sequences were identified in the proximal 3′ untranslated regions of each gene product except MS4A6A, MS4A6E, MS4A10, and MS4a6C. Two poly(A) signal sequences were found in MS4a4D, MS4A5, and MS4a10 transcripts, while four were observed in MS4A4A transcripts.

The disclosed MS4A cDNAs were further used to annotate the genomic sequence derived from BAC clones. Annotated features include definition of coding regions, intronlexon junctions, sequences upstream of the initial coding region of each gene that comprise the promoter region, and other adjacent sequences that could also comprise gene regulatory elements. Representative methods for further characterizing a MS4A promoter region are disclosed in Example 9.

Annotation of human MS4A genomic regions (SEQ ID NOs:73-81), as disclosed herein, enabled a comparison of gene structure among MS4A genes. The overall domain organization of each MS4A gene was similar (FIGS. 2 and 7). All exonlintronlexon boundaries were consistent with consensus splice-donor and -acceptor sequences unless otherwise indicated, with exon|GTGAGT-intron-CAG|exon sequences in most cases (Aebi & Weissmann, 1987). In addition, the splice junctions for all translated exons were located after the third nucleotide in each codon. Most MS4A proteins were encoded by 6 exons except MS4A2, MS4A5, and MS4A6E (FIGS. 2 and 7). In these exceptions: the N-terminal cytoplasmic domain of MS4A2 was encoded by two exons. (Kuster et al., 1992); the MS4A5 and MS4A6E genes did not encode C-terminal cytoplasmic domains; and the MS4A6E gene had only two membrane spanning domains. Intron lengths demonstrated wide variation from 181 bp in MS4A12 to 13,731 bp in MS4A5. In some cases however, exact intron lengths were not determined; MS4A3, MS4A4, and MS4A12 (FIG. 2). Distances between translation initiation and termination codons were determined for most MS4A genes; with MS4A6E being the smallest (5,060 bp) and MS4A4E being the longest (23,379 bp) genes (FIG. 6). Thus, the intronlexon organization of all MS4A family members is consistent with the high degree of conservation within this gene family.

There were no amino-terminal signal sequences, although all MS4A proteins contained hydrophobic regions of sufficient length to pass through the membrane at least four times. Notable was a marked clustering of charged residues at both ends of the putative transmembrane domains, some of which were highly conserved. In some cases, the first and second putative transmembrane domains of MS4A proteins were a continuous stretch of hydrophobic amino acids without an obvious inter-transmembrane hydrophilic bridge. By contrast, MS4A4A and MS4A7 had 6 to 7 hydrophilic amino acids inserted between the first and second hydrophobic domains. In human MS4A4A and mouse MS4a4B, MS4a4C, and MS4a4D, an extensive hydrophobic region followed the fourth putative membrane-spanning domain. Thus, the overall structure of MS4A family members was well conserved.

II.E. MS4A Gene Splice Variants

Among the MS4A cDNAs sequenced and EST sequences analyzed, multiple splice variants were identified that encoded variant MS4A proteins. In most cases, exons were spliced out, which generated truncated protein products. Potential splice variants of the MS4A4A, MS4A5, MS4A6A, and MS4A7 genes were identified. Whether these alternatively spliced variants produce functional proteins has yet to be determined.

Two splice variations of the MS4A4A gene were identified during an analysis of MS4A4A mRNA expression by lymphoblastoid cell lines. Most of the hematopoietic cell lines examined expressed transcripts encoding a full-length MS4A4A protein as shown in FIG. 7. However, a second smaller transcript was also expressed in most cases that contained a potential exon deletion of 158 nucleotides. This was a frequent event since 40% of MS4A4A cDNAs generated from the BJAB B cell line encoded the truncated protein. In addition, the same splicing event was observed in two of five EST sequences that covered this region of the MS4A4A protein. Splicing-out this potential exon deleted the third membrane-spanning domain and the second extracellular loop from the full-length protein (positions 110-163, FIG. 3). Of interest, this splicing event fused the first/second membrane spanning domains with the fourth membrane spanning domain. However, the fourth transmembrane spanning domain in MS4A4A is followed by another hydrophobic region of sufficient length to traverse the membrane (disclosed herein). This suggests that differential splicing can generate an alternative MS4A4A protein with four membrane spanning domains lacking a significant extracellular domain.

In the case of the MS4A5 gene, two of nine MS4A5 EST sequences analyzed (GenBank Accession Nos. M411806 and AA781801) encoded a splice variant that preserved the reading frame of the transcript. In both sequences, the exon encoding the third membrane-spanning domain and the second extracellular loop from the full-length protein (TM3, FIG. 1) was spliced out using normal splice-donor and -acceptor sequences, which deleted 51 amino acids (114-164) from the full length protein (FIG. 7). This deletion resulted in a protein with the first/second membrane spanning domains fused with the fourth predicted membrane-spanning domain. Thus, the truncated MS4A5 protein would possess three membrane-spanning domains with an extracellular carboxyl-terminal domain.

A novel splicing event was observed in the MS4A6A gene which resulted in a truncated protein. A novel splice donor site (CAG T⁶⁸³|GT GAG T) is located within the exon encoding the TM3/extracellular loop domains (FIG. 4). This cryptic splice donor site was spliced with the normal 3′ splice acceptor site of the exon encoding the TM4 domain, which thereby deletes nucleotides 684-787 from MS4A6A transcripts (FIG. 4). Since there was an extra T introduced into the codon sequence due to this alternative splicing event, there was a frameshift in the coding sequence. This potentially results in the attachment of a novel 30 amino acid sequence (—WNSLSDADLHSAGILPSCAHCCMVETGLL) that is not predicted to be hydrophobic. Thus, the variant MS4A protein would be 70 amino acids shorter and would lack the fourth membrane-spanning and cytoplasmic domains. This alternative splicing event was found in 3 of 29 EST sequences that encoded this region (GenBank Accession Nos. A1278475, AA461046, and AA448335) and in one cDNA clone (GenBank Accession No. AB013104).

Splice variation in MS4A7A transcripts produces two distinct protein products in addition to the presumably normal protein. In one case, a splice variation in MS4A7A transcripts produces a protein product similar in structure to the MS4A6E protein. The exon encoding the first/second membrane spanning domains (amino acids 50-94, FIG. 7) was deleted in 2 of 4 MS4A7 EST sequences analyzed (GenBank Accession Nos. N42191 and R11179) that cover this region. Thus, the protein product would have a longer N-terminal cytoplasmic domain and only two membrane spanning domains. In the second case, the exon encoding the fourth membrane-spanning domain (amino acids 183-216) was deleted in 2 EST sequences (GenBank Accession Nos. R11180 and A1188478) out of 18 sequences analyzed (FIG. 7).

II.F. MS4A Gene Polymorphisms

Putative polymorphisms were identified in the MS4A6A gene. Two nucleotide substitutions were found in cDNA clone ATCC No. 499181 and in 13 of 38 EST sequences analyzed (FIG. 1). The first substitution was at nucleotide 373 that exchanged a C for a T, which did not alter the amino acid sequence. The second substitution resulted in a Ser in place of Thr at amino acid 185. In addition, a third substitution was found in 4 of the 38 EST sequences analyzed where a Ser was substituted in place of an Ala at amino acid position 183. This substitution was paired with a Ser to Thr substitution at amino acid position 185 in half of the clones analyzed. These differences most likely represent common sequence polymorphisms since they were observed in multiple independent cDNA clones. Based on our genetic DNA analysis, it is unlikely that these differences could represent transcripts from distinct genes that are almost identical in coding sequence.

As with the MS4A6A gene (disclosed herein), potential gene polymorphisms were observed in MS4A6E. Three cDNA clones representing partial transcripts were sequenced completely on both strands. The predicted MS4A6E gene product and one cDNA clone (ATCC No. 3704466) had identical sequences. However, the ATCC No. 3557769 cDNA had a nucleotide substitution at position 314 (FIG. 4) that exchanged a T for a C, which did not alter the predicted amino acid sequence. The ATCC No. 1852248 cDNA clone had the longest insert that starts at nucleotide position 60 and ended at position 661 as shown in FIG. 4. This cDNA had a substitution at nucleotide 153 that exchanged a G for a T, which resulted in a Phe in place of Val at amino acid 47 (FIG. 4). Therefore, sequence polymorphisms can exist within the MS4A6E gene.

Other potential polymorphisms were observed in other MS4A family members based on consistent nucleotide variations found in MS4A4E sequences.

The assembly and annotation of genomic sequences comprising MS4A genes in the region of human chromosome 11q12-13.1, disclosed herein for the first time, provide source material for identification of polymorphisms that are linked to MS4A genes. Such polymorphisms can include single nucleotide polymorphisms as disclosed within the MS4A6A and MS4A6E coding region sequences. In addition, polymorphisms within or genetically linked to MS4A genes can also comprise restriction length polymorphisms (RFLPs) (Lander & Botstein (1989) Genetics 121:185-199), short tandem repeat polymorphisms (STRPs), short sequence length polymorphisms (SSLPs) (Dietrich et al. (1996) Nature 380:149-152), amplified fragment length polymorphisms (AFLPs) (Latorra et al. (1994) PCR Methods Appl 3(6):351-358), and microsatellite markers (Schalkwyk et al. (1999) Genome Res 9:878-887). Identification of polymorphisms within an isolated DNA molecule are known to one of skill in the art.

II.G. MS4A Proteins

The MS4A genes encoded proteins of 16-29 kDa (Table 2).

TABLE 2
MS4A Family Members

| Human | | Mouse | | Human/Mouse |
|---|---|---|---|---|
| Name | kDa | Name | kDa | Homology |
| | | MS4a3 | | 63% (partial) |
| MS4A4A | 23 | | | |
| | | Ms4a4B | 24 | 41% |
| | | Ms4a4C | 24 | 44% |
| | | Ms4a4D | 24 | 40% |
| MS4A4E | 24 | | | |
| MS4A5 | 22 | | | |
| MS4A6A | 27 | | | |
| | | Ms4a6B | 27 | 52% |
| | | Ms4a6C | 24 | 51% |
| | | Ms4a6D | 26 | 53% |
| MS4A6E | 16 | | | |
| MS4A7 | 26 | MS4a7 | 26 | 53% |
| MS4A8B | 26 | MS4a8B | 29 | 63% |
| MS4A10 | 27 | MS4a10 | 29 | 52% |
| MS4A12 | 26 | MS4a12_{(pig)} | 26 | 60% |

^{a}Predicted molecular weights for the new MS4A family members and the percentage amino acid sequence identity between deduced MS4A and MS4a proteins.

Comparisons between CD20 and the predicted amino acid sequences for human MS4A4A, MS4A5, MS4A6A, MS4A7, MS4A8B, and MS4A12 revealed 23-29% amino acid sequence identity (FIG. 7). The highest degree of identity was found in the first three transmembrane domains with multiple regions of conserved amino acids. In particular, the amino acid sequences LGAXQI (SEQ ID NO:57) and LSLG (SEQ ID NO:58) were common within the first transmembrane domain, GYPFWG (SEQ ID NO:60) and FIISGSLS (SEQ ID NO:61) were common in the second domain, and SLX₂NX₂SX₃AX₂G (SEQ ID NO:62) was found in the third transmembrane domain. The first and second transmembrane domains of MS4A8B were 46% identical in amino acid sequence with human CD20, 41% identical with FcεRIβ, and 39% identical with HTm4. The MS4A4A, MS4A5, MS4A6A, and MS4A7 proteins were most homologous in their first and second transmembrane domains with the human FcεRIβ chain, with 37-46% amino acid sequence identity. There was large variation between MS4A proteins in the N- and C-terminal cytoplasmic domains. However, Pro residues were significantly over-represented within the N- and C-terminal cytoplasmic domains of most MS4A family members. There was some sequence identity in the first potential extracellular loop that was ˜13 amino acids in length for each protein. By contrast, the second predicted extracellular loop ranged from 10-46 amino acids in length with diverse sequences.

The putative MS4A4E gene encodes a 220 amino acid protein of 23.8 kDa with a predicted amino acid sequence that is 76% identical with the MS4A4A protein (FIG. 3). Consistent with other MS4A proteins, the most significant homologies between MS4A4E and other MS4A family members were found in the membrane spanning domains (FIG. 7). Common amino acid motifs were readily visualized such as KXLGAIQI (SEQ ID NO:57), GYPXWG (SEQ ID NO:60), and SGXLSI (SEQ ID NO:59) in the first and second hydrophobic regions that represent potential transmembrane regions. The intracellular N- and C-terminal domains were highly conserved between MS4A4E and MS4A4A, but were divergent from other family members.

The putative MS4A6E gene encodes a 147 amino acid protein of 15.9 kDa with a predicted amino acid sequence that is 78% identical with the MS4A6A protein (FIG. 4). The most significant homologies between MS4A6E and other MS4A family members were found in the membrane spanning domains, although MS4A6E only had two (TM3 and TM4) membrane-spanning domains (FIGS. 4 and 7). The putative second extracellular loops of MS4A6E and MS4A6A were of identical length (FIG. 4). Common amino acid motifs were readily visualized in the hydrophobic regions that represent potential transmembrane regions. The intracellular N-terminal domain was highly conserved between MS4A6E and MS4A6A, but were divergent from other family members. MS4A6E protein also lacks a C-terminal cytoplasmic domain (FIG. 4).

The putative MS4A10 gene encodes a translated 241 amino acid protein of 26.9 kDa with a predicted amino acid sequence that is 52% identical with the mouse MS4A10 protein (FIG. 5). The most significant homologies between MS4A10 and MS4A10 were found in the membrane spanning domains and the putative second extracellular loop (FIG. 5). Although the N-terminal cytoplasmic domains of MS4A10 and MS4A10 were of similar length, the intracellular N- and C-terminal domains had the lowest sequence homologies among domains. The cytoplasmic C-terminal domain was 28 amino acids shorter in MS4A10 than MS4A10. Nonetheless, based on the sequence similarities of translated regions, it appears that MS4A10 and MS4A10 represent homologous genes that are more similar to one another than other MS4A family members.

Ten novel mouse MS4A proteins were identified that shared 40-63% amino acid sequence identity with their potential human counterparts (FIG. 7, Table 2). For comparison, the mouse and human CD20 proteins are 74% identical in amino acid sequence (Tedder et al., 1988a). A single partial cDNA was identified that encoded the mouse homologue for HTm4 (MS4a3, FIG. 7). The predicted amino terminus of the proposed MS4a3 protein was 23 amino acids shorter than in the human protein, although their overlapping regions were 63% identical in amino acid sequence. In all cases, the transmembrane domains of the human and mouse MS4A proteins were the most well conserved regions. For example, the human MS4A8B protein was 78% identical in sequence to MS4a8B in the first 3 transmembrane domains and 68% identical in domain 4. Additional MS4A genes are likely to be identified in humans and mice, including the mouse MS4A5 homologue.

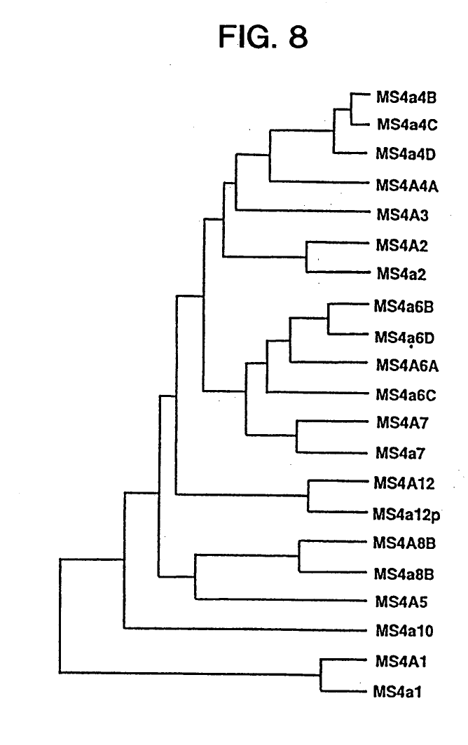

A UPGMA (unweighted pair group method using arithmetic averages) tree showing relatedness of deduced MS4A and MS4a protein sequences is depicted in FIG. 8.

III. Methods for Detecting a MS4A Nucleic Acid Molecule

In another aspect of the invention, a method is provided for detecting a nucleic acid molecule that encodes a MS4A polypeptide. According to the method, a biological sample having nucleic acid material is procured and hybridized under stringent hybridization conditions to a MS4A nucleic acid molecule of the present invention. Such hybridization enables a nucleic acid molecule of the biological sample and the MS4A nucleic acid molecule to form a detectable duplex structure. Preferably, the MS4A nucleic acid molecule includes some or all nucleotides of any one of the odd-numbered SEQ ID NOs:1-37. Also preferably, the biological sample comprises human nucleic acid material.

III.A. Expression of MS4A Family Members in Hematopoietic Cells

Since CD20, FcεRIβ, and HTm4 expression are restricted to hematopoietic tissues, MS4A gene transcription was assessed by PCR amplification of cDNA from eleven human hematopoietic cell lines. Like CD20, MS4A8B was only expressed by B cell lines (Table 3). MS4A5 was only expressed by a promonocytic cell line. MS4A6A transcripts were expressed by B cell, myelomonocytic, and erythroleukemia cell lines. MS4A4A mRNA was expressed by all cell lines examined, although the relative mRNA levels varied significantly. MS4A7 was expressed in most, but not all of the cell lines tested. MS4A12 transcripts were not detected in these cell lines. Thus, most MS4A family members are likely to be expressed in hematopoietic tissues.

ESTs encoding MS4A transcripts were isolated from a variety of different cDNA libraries. MS4A4A ESTs were from aorta, brain, breast, heart, kidney, lung, ovary, pancreas, placenta, prostate, stomach, testis, and uterine tissues. MS4A5 ESTs were only isolated from testis. MS4A6A ESTs were from aorta, brain, the central nervous system, colon, gall bladder, heart, kidney, lung, muscle, ovary, pancreas, placenta, prostate, skin, stomach, tonsil, uterus and embryonic tissues. MS4A7 ESTs were from lung, kidney, lymphocytes, mammary gland, placenta, spleen, testis, thymus, and uterine tissues. MS4A8B ESTs were from brain, lung, uterus and embryonic tissues. A single MS4A12 EST was isolated from colon. This demonstrates differential MS4A gene transcription among lymphoid and non-lymphoid tissues.

TABLE 3
MS4A mRNA Expression by Human Lymphoblastoid Cell Lines
MS4A family member^{a}

| | | | | | | | | | | G3- |
|---|---|---|---|---|---|---|---|---|---|---|
| Cell lines: | 1 | 2 | 3 | 4A | 5 | 6A | 7 | 8B | 12 | PDH |
| Pre-B: | | | | | | | | | | |
| NALM-6 | − | − | − | +++ | − | − | − | − | − | +++ |
| B cell: | | | | | | | | | | |
| BJAB | +++ | − | − | +++ | − | − | +++ | + | − | +++ |
| DAUDI | +++ | − | − | + | − | − | +++ | + | − | +++ |
| SB | +++ | − | − | ++ | − | +++ | +++ | + | − | +++ |
| T cell: | | | | | | | | | | |
| HSB-2 | − | − | − | + | − | − | − | − | − | +++ |
| HUT-78 | − | − | − | + | − | − | + | − | − | +++ |
| JURKAT | − | − | − | + | − | − | − | − | − | +++ |
| MOLT15 | − | − | − | + | − | − | ++ | − | − | +++ |

Myelomonocyte:

| | | | | | | | | | | |
|---|---|---|---|---|---|---|---|---|---|---|
| HL60 | − | − | +++ | ++ | − | +++ | +++ | − | − | +++ |
| U937 | − | − | +++ | +++ | + | + | +++ | − | − | +++ |

Erythroleukemia:

| K562 | − | + | +++ | +++ | − | + | − | − | − | +++ |
|---|---|---|---|---|---|---|---|---|---|---|
| ^{a}Gene transcription was assessed by PCR amplification of cDNA generated from mRNA isolated from each cell type. Values represent the level of PCR product generated relative to the glyceraldehyde−3−phosphate dehydrogenase (G3PDH) control in three separate PCR reactions: −, no specific PCR product detected; +, low levels of the appropriate band were detectable; ++ to +++, appropriate bands of increasing intensity were readily visualized in all samples | | | | | | | | | | |
| # examined. Identical results were obtained using two different primer pairs for cDNA amplification. | | | | | | | | | | |

Since most of the MS4A genes are expressed by hematopoietic cells, MS4A4E, MS4A6E and MS4A10 transcription were assessed by RT-PCR amplification of cDNA from human hematopoietic cell lines and human tissues. Transcripts from eleven human hematopoietic cell lines were evaluated; one pre-B cell line (NALM-6), three B cell lines (BJAB, DAUDI, and SB), four T cell lines (HSB-2, HUT-78, JURKAT, and MOLT15), two myelomonocytic lines (HL60 and U937), and one erythroleukemia cell line (K562). In addition, transcripts from eight human tissues were evaluated; colon, ovary, peripheral blood leukocytes, prostate, small intestine, spleen, testes and thymus. However, MS4A4E, MS4A6E and MS4A10 transcripts were not detected in any of these cell lines or tissues.

MS4A4E, MS4A6E, and MS4A10 sequences were also used to search the translated GenBank databases using the BLAST program (Altschul et al., 1997). Eleven EST sequences representing MS4A6E transcripts were found that represented nine cDNAs isolated from pooled fetal organ libraries (GenBank Accession Nos. AA382998, AA909515, M917066, A1222355, A1279944, A1684553, A1699419, A1743473, A1806247), one cDNA from a pooled germ cell tumor library (GenBank Accession No. A1968835), and one cDNA from a colon tumor (GenBank Accession No. AW951636). EST cDNAs encoding MS4A4E or MS4A10 sequences were not identified. This suggests that MS4A4E, MS4A6E, and MS4A10 transcripts are rare among normal tissues or they are primarily expressed during oncogenesis or embryogenesis.

MS4a gene expression by mouse tissues was assessed by Northern analysis and PCR amplification of cDNAs (Table 4). In most cases assessed, Northern analysis failed to detect specific MS4a transcripts in tissues that revealed transcript production by PCR amplification. These results suggest that MS4a transcripts are only produced by subpopulations of cells within each tissue such that transcript levels were often below the level of detection by Northern analysis. Nonetheless, MS4a4B, MS4a4C, and MS4a6B transcripts were found at high levels in thymus, spleen and peripheral lymph nodes, with less abundant levels in non-lymphoid tissues. MS4a6C was only expressed by thymus, spleen, PLN and bone marrow. MS4a4C, MS4a6D and MS4a7 were expressed in all tissues examined. MS4a8B transcripts were expressed by spleen, peripheral lymph nodes, colon, liver, heart, lung and bone marrow. MS4A10 transcripts were found in thymus, kidney, colon, brain, and testis. In addition, CD20 (MS4a1), FcεRIβ (MS4a2), and MS4a3 expression were primarily restricted to hematopoietic tissues. MS4a3, MS4a4B, MS4a4C, MS4a6B, MS4a6C, MS4a6D, MS4a7, MS4a8B, and MS4A10 were also expressed by various hematopoietic and lymphoblastoid cell lines. Therefore, most MS4a family members were expressed by hematopoietic cells.

TABLE 4
MS4a Gene Expression by Mouse Tissues^{a}

| MS4a | Thymus | Spleen | PLN | BM | Liver | Kidney | Heart | Colon | Lung | Brain | Testes |
|---|---|---|---|---|---|---|---|---|---|---|---|
| 1 | + | +++ | +++ | + | − | − | − | − | + | − | − |
| 2 | + | + | + | +++ | − | + | − | − | + | − | − |
| 3 | + | + | + | +++ | − | − | − | − | + | + | − |
| 4B | +++ | +++ | +++ | ++ | + | + | + | + | + | − | − |
| 4C | +++ | +++ | +++ | +++ | + | + | + | + | + | + | + |
| 4D | + | + | ++ | − | + | + | ++ | ++ | ++ | − | + |
| 6B | +++ | +++ | +++ | ++ | + | − | + | + | + | − | ++ |
| 6C | + | + | + | ++ | − | − | − | − | − | − | − |
| 6D | +++ | +++ | +++ | ++ | +++ | +++ | +++ | +++ | +++ | +++ | +++ |
| 7 | ++ | ++ | ++ | ++ | + | + | + | ++ | + | + | + |
| 8B | − | + | + | + | + | − | + | ++ | + | − | − |
| 10 | + | − | − | − | − | + | − | + | − | + | ++ |
| G3PDH | +++ | +++ | +++ | +++ | +++ | +++ | +++ | +++ | +++ | +++ | +++ |

^{a}Gene transcription was assessed by PCR amplification of cDNA generated from mRNA isolated from tissue samples. Values represent the level of PCR product generated relative to the glyceraldehyde-3-phosphate dehydrogenase (G3PDH) control as described for Table 3. Peripheral lymph node (PLN) and bone marrow (BM).

Expression of MS4A family members was also assessed in mouse hematopoietic cell lines (Table 5). Nine of the twelve MS4A genes were expressed in pre-B cell lines and five of the MS4A genes were expressed in B cell lines. Six of the MS4A genes were expressed by T cell lines. These data suggest that B cells can express most members of the MS4A gene family, although the patterns of expression of each gene is distinct.

TABLE 5
MS4a Expression by Mouse Lymphoid Tissues and Cell Lines^{a}

| | Tissues | | Pre B cell lines | | | B cell lines | | T cell lines | |
|---|---|---|---|---|---|---|---|---|---|
| MS4a | Spleen | Thymus | 300.19 | 38B9 | 70Z | A20 | AJ9 | BW514 | EL-14 |
| 1 | +++ | + | − | − | − | +++ | +++ | − | − |
| 2 | + | + | − | − | + | − | − | − | − |
| 3 | + | + | − | + | − | − | − | − | − |
| 4B | +++ | +++ | − | − | − | − | − | − | ++ |
| 4C | +++ | +++ | − | + | + | ++ | − | − | + |
| 4D | + | + | − | − | − | − | − | − | − |
| 6B | +++ | +++ | − | + | +++ | + | − | +++ | +++ |
| 6C | + | − | − | + | + | − | − | − | + |
| 6D | +++ | +++ | − | ++ | +++ | − | + | − | +++ |
| 7 | ++ | ++ | − | − | ++ | − | − | − | − |
| 8B | + | − | − | − | ++ | − | − | − | − |
| 10 | − | + | − | − | + | + | + | + | − |
| G3PDH | +++ | +++ | +++ | +++ | +++ | +++ | +++ | +++ | +++ |
| ^{a}Gene transcription was assessed by PCR amplification of cDNA generated from mRNA isolated from each cell type. Values represent the level of PCR product generated relative to the glyceraldehyde-3-phosphate dehydrogenase (G3PDH) control in three separate PCR reactions: −, no specific PCR product detected; +, low levels of the appropriate band were detectable; ++ to +++, appropriate bands of increasing intensity were readily visualized in all samples examined. | | | | | | | | | |
| # Identical results were obtained using two different primer pairs for cDNA amplification. | | | | | | | | | |

III.B. Detection of MS4A Polymorphisms

In another embodiment, genetic assays based on nucleic acid molecules of the present invention can be used to screen for genetic variants by a number of PCR-based techniques, including single-strand conformation polymorphism (SSCP) analysis (Orita, M., et al. (1989) Proc Natl Acad Sci USA 86(8):2766-2770), SSCP/heteroduplex analysis, enzyme mismatch cleavage, and direct sequence analysis of amplified exons (Kestila et al. (1998) Mol Cell 1(4):575-582; Yuan et al. (1999) Hum Mutat 14(5):440-446). Automated methods can also be applied to large-scale characterization of single nucleotide polymorphisms (Brookes (1999) Gene 234(2):177-186; Wang et al. (1998) Science 280(5366):1077-82). The present invention further provides assays to detect a mutation of a variant MS4A locus by methods such as allele-specific hybridization (Stoneking et al. (1991) Am J Hum Genet 48(2):370-82), or restriction analysis of amplified genomic DNA containing the specific mutation.

IV. Recombinant Production of a MS4A Polypeptide

The present invention also provides a method for recombinant production of a MS4A polypeptide, as described in Example 3. Preferably, the recombinant polypeptide comprises some or all of the amino acid sequences of any one of the even-numbered SEQ ID NOs:2-38.

Recombinantly produced proteins are useful for a variety of purposes, including structural determination of a MS4A polypeptide, generation of an antibody that recognizes a MS4A polypeptide, and screening assays to identify a chemical compound or peptide that interacts with a MS4A polypeptide, described further herein below.

V. Production of MS4A Antibodies

In another aspect, the present invention provides a method of producing an antibody immunoreactive with a MS4A polypeptide, the method comprising recombinantly or synthetically producing a MS4A polypeptide, or portion thereof, to be used as an antigen. The MS4A polypeptide is formulated so that it is can be used as an effective immunogen. An animal is immunized with the formulated MS4A polypeptide, generating an immune response in the animal. The immune response is characterized by the production of antibodies that can be collected from the blood serum of the animal. Optionally, cells producing a MS4A antibody can be fused with myeloma cells, whereby a monoclonal antibody can be selected. Exemplary methods for producing a monoclonal antibody that recognizes a MS4A protein are described in Example 4. Preferred embodiments of the method use a polypeptide set forth as any one of the even-numbered SEQ ID NOs:2-38.

The present invention also encompasses antibodies and cell lines that produce monoclonal antibodies as described herein.

The foregoing antibodies can be used in methods known in the art relating to the localization and activity of the MS4A polypeptide sequences of the invention, e.g., for cloning of MS4A nucleic acids, immunopurification of MS4A polypeptides, imaging MS4A polypeptides in a biological sample, measuring levels thereof in appropriate biological samples, and in diagnostic methods.

VI. Methods for Detecting a MS4A Polypeptide

In another aspect of the invention, a method is provided for detecting a level of MS4A polypeptide using an antibody that specifically recognizes a MS4A polypeptide, or portion thereof. In a preferred embodiment, biological samples from an experimental subject and a control subject are obtained, and MS4A polypeptide is detected in each sample by immunochemical reaction with the MS4A antibody. More preferably, the antibody recognizes amino acids of any one of the even-numbered SEQ ID NOs:2-38, and is prepared according to a method of the present invention for producing such an antibody.

In one embodiment, a MS4A antibody is used to screen a biological sample for the presence of a MS4A polypeptide. A biological sample to be screened can be a biological fluid such as extracellular or intracellular fluid, or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide. In accordance with a screening assay method, a biological sample is exposed to an antibody immunoreactive with a MS4A polypeptide whose presence is being assayed, and the formation of antibody-polypeptide complexes is detected. Techniques for detecting such antibody-antigen conjugates or complexes are well known in the art and include but are not limited to centrifugation, affinity chromatography and the like, and binding of a labeled secondary antibody to the antibody-candidate receptor complex.

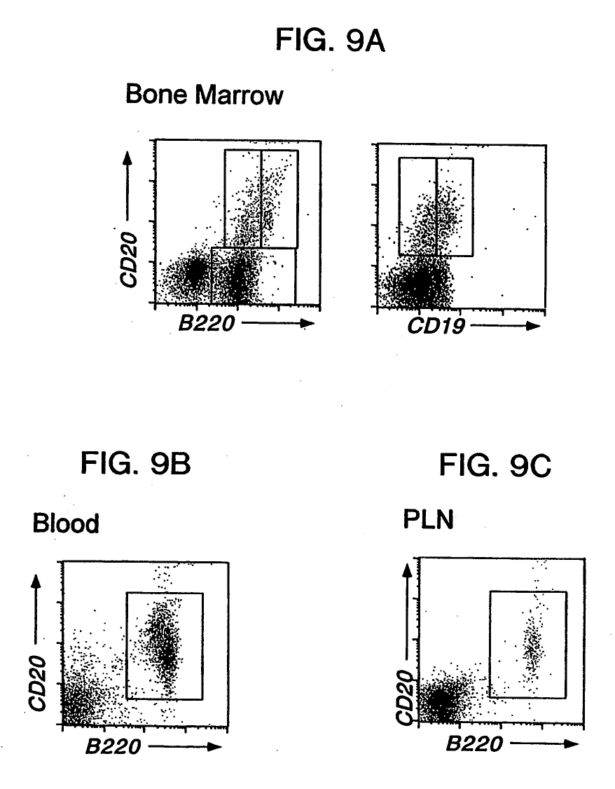

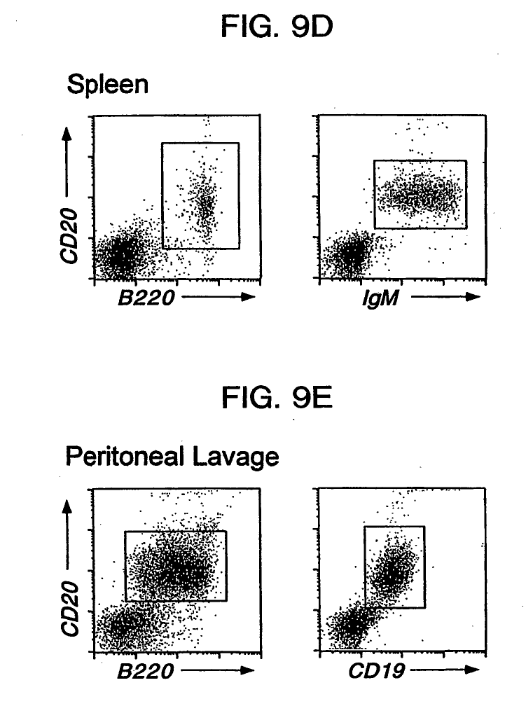

In one embodiment, an antibody that specifically recognizes a MS4A polypeptide can be used to assess the tissue- or cell-distribution of MS4A protein, for example, to evaluate CD20 expression during B lymphocyte development (FIG. 9). CD20 expression in B220+lymphocytes from lymphoid tissues of wild type mice was examined by two-color immunofluorescence. In bone marrow, three types of B220⁺ cells were detected. The vast majority of B220^{hi} lymphocytes expressed CD20. However, the majority of B220^{lo} lymphocytes were CD20-negative. Thus, CD20 was predominantly expressed by mature B cells.

CD19 expression is restricted to normal and neoplastic B cells and follicular dendritic cells. CD19 is expressed early by B progenitor cells in the bone marrow, presumably at the late pro-B or early pre-B cell stages around the time of immunoglobulin heavy chain rearrangement (Anderson et al. (1984) Blood 63:1424). Expression persists during all stages of B cell maturation and is lost upon terminal differentiation to plasma cells.

Double staining of CD20 with IgM and CD19 antibodies showed that some of the CD19^{lo} and IgM^{lo} cells were CD20 negative in the bone marrow. A few IgM- cells also expressed low levels of CD20 in the bone marrow. This data suggested that the CD20 expression was later than the CD19 expression but before or around the time of IgM expression during B cell development in the bone marrow since these cells were gated on lymphocytes not dendritic cells.

The level of CD20 expression observed on mature B220^{hi} B cells in bone marrow was maintained by B cells from peripheral lymphoid tissues. The vast majority of B220⁺ B cells in the spleen, blood, peripheral lymph nodes, and peritoneal cavity expressed CD20. Therefore, like human CD20, mouse CD20 was also exclusively expressed on B cells from the immature B cell stage to mature B cells.

VII. Identification of MS4A Modulators

VII.A. Screening for Small Molecule Ligands that Interact with a MS4A Polypeptide

The present invention further discloses a method for identifying a compound that modulates MS4A function. According to the method, a MS4A polypeptide is exposed to a plurality of compounds, and binding of a compound to the isolated MS4A polypeptide is assayed. A compound is selected that demonstrates specific binding to the isolated MS4A polypeptide. Preferably, the MS4A polypeptide used in the binding assay of the method includes some or all amino acids of any one of the even-numbered SEQ ID NOs:2-38.

Several techniques can be used to detect interactions between a protein and a chemical ligand without employing an in vivo ligand. Representative methods include, but are not limited to, Fluorescence Correlation Spectroscopy, Surface-Enhanced Laser Desorption/Ionization Time-Of-flight Spectroscopy, and Biacore technology, as described in Example 5. These methods are amenable to automated, high-throughput screening.

Candidate regulators include but are not limited to proteins, peptides, and chemical compounds. Structural analysis of these selectants can provide information about ligand-target molecule interactions that enable the development of pharmaceuticals based on these lead structures.

Similarly, the knowledge of the structure a native MS4A polypeptide provides an approach for rational drug design. The structure of a MS4A polypeptide can be determined by X-ray crystallography or by computational algorithms that generate three-dimensional representations. See Huang et al. (2000) Pac Symp Biocomput 23041; Saqi et al. (1999) Bioinformatics 15:521-522. Computer models can further predict binding of a protein structure to various substrate molecules, that can be synthesized and tested. Additional drug design techniques are described in U.S. Pat. Nos. 5,834,228 and 5,872,011.

VII.B. Methods for Identifying Modulators of MS4A Gene Expression

The assembly and annotation of genomic sequences comprising MS4A genes in the region of human chromosome 11q12-13.1, disclosed herein for the first time, identify MS4A gene regulatory regions. Preferably, MS4A gene regulatory regions comprise sequences upstream of the initial coding region of each MS4A gene as disclosed in SEQ ID NOs:73-81. An expression cassette comprising a MS4A promoter region can be employed in assays for the identification of modulators of MS4A expression. Thus the present invention also provides a method for identifying a substance that regulates MS4A gene expression using a chimeric gene that includes an isolated MS4A gene promoter region operably linked to a reporter gene. According to this method, a gene expression system is established that includes the chimeric gene and components required for gene transcription and translation so that reporter gene expression is assayable. To select a substance that regulates MS4A gene expression, the method further provides the steps of using the gene expression system to determine a baseline level of reporter gene expression in the absence of a candidate regulator; providing one or more candidate regulators to the gene expression system; and assaying a level of reporter gene expression in the presence of a candidate regulator. A candidate regulator is selected whose presence results in an altered level of reporter gene expression when compared to the baseline level.

Several molecular cloning strategies can be used to identify substances that specifically bind a MS4A gene cis-regulatory element. In one embodiment, a cDNA library in an expression vector, such as the lambda-gt11 vector, can be screened for cDNA clones that encode a MS4A gene regulatory element DNA-binding activity by probing the library with a labeled MS4A DNA fragment, or synthetic oligonucleotide (Singh et al. (1989) Biotechniques 7:252-261). Preferably, the nucleotide sequence selected as a probe has already been demonstrated as a protein binding site using a protein-DNA binding assay, as described in Example 9.

In another embodiment, transcriptional regulatory proteins are identified using the yeast one-hybrid system (Luo et al. (1996) Biotechniques 20(4):564-568; Vidal et al. (1996) Proc Natl Acad Sci USA 93(19):10315-10320; Li & Herskowitz (1993) Science 262:1870-1874). In this case, a cis-regulatory element of a MS4A gene is operably fused as an upstream activating sequence (UAS) to one, or typically more, yeast reporter genes such as the lacZ gene, the URA3 gene, the LEU2 gene, the HIS3 gene, or the LYS2 gene, and the reporter gene fusion construct(s) is inserted into an appropriate yeast host strain. It is expected that the reporter genes are not transcriptionally active in the engineered yeast host strain, for lack of a transcriptional activator protein to bind the UAS derived from the MS4A gene promoter region. The engineered yeast host strain is transformed with a library of cDNAs inserted in a yeast activation domain fusion protein expression vector, e.g. pGAD, where the coding regions of the cDNA inserts are fused to a functional yeast activation domain coding segment, such as those derived from the GAL4 or VP16 activators. Transformed yeast cells that acquire a cDNA encoding a protein that binds a cis-regulatory element of a MS4A gene can be identified based on the concerted activation the reporter genes, either by genetic selection for prototrophy (e.g. LEU2, HIS3, or LYS2 reporters) or by screening with chromogenic substrates (e.g., a lacZ reporter) by methods known in the art.

The present invention also provides an in vivo assay for discovery of modulators of MS4A gene expression. In this case, a transgenic non-human animal is made such that a transgene comprising a MS4A gene promoter and a reporter gene is expressed and a level of reporter gene expression is assayable. Such transgenic animals can be used for the identification of compounds that are effective in modulating MS4A gene expression. In vitro or in vivo screening approaches can also survey more than one modulatable transcriptional regulatory sequence simultaneously.

VIII. Animal Models

The present invention further pertains to an animal model of disorders associated with a MS4A nucleic acid or polypeptide, including but not limited to atopic disorders, abnormal target cell development, function, and Ca⁺⁺ responses. Such a model can be prepared by several methods. Using a transgenic approach, knock-out, knock-in, or knock-down mutation of the MS4A gene can suppress MS4A function. The present invention also teaches that an animal model of a MS4A-related disorder can be prepared by immunizing an animal with a MS4A polypeptide. The resulting immune response in the animal comprises a production of antibodies that specifically bind a MS4A polypeptide, thereby disrupting its biological activity. A method is also provided for generating an animal model of a MS4A-related disorder by administering to an animal a compound that disrupts MS4A expression or function. Such a compound is discovered by methods disclosed herein.

VIII.A. Generation of CD20-Deficient Mice

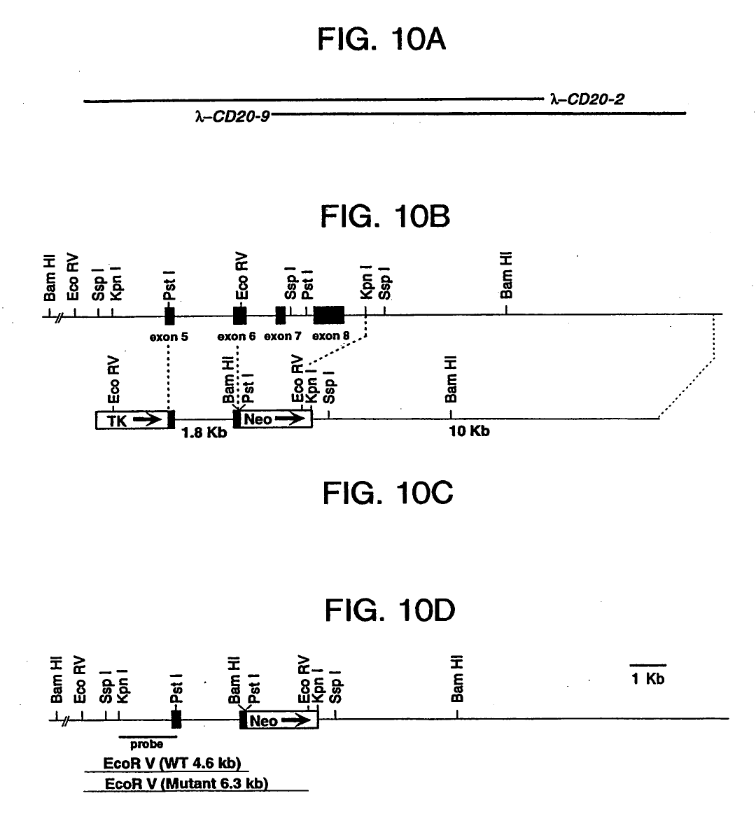

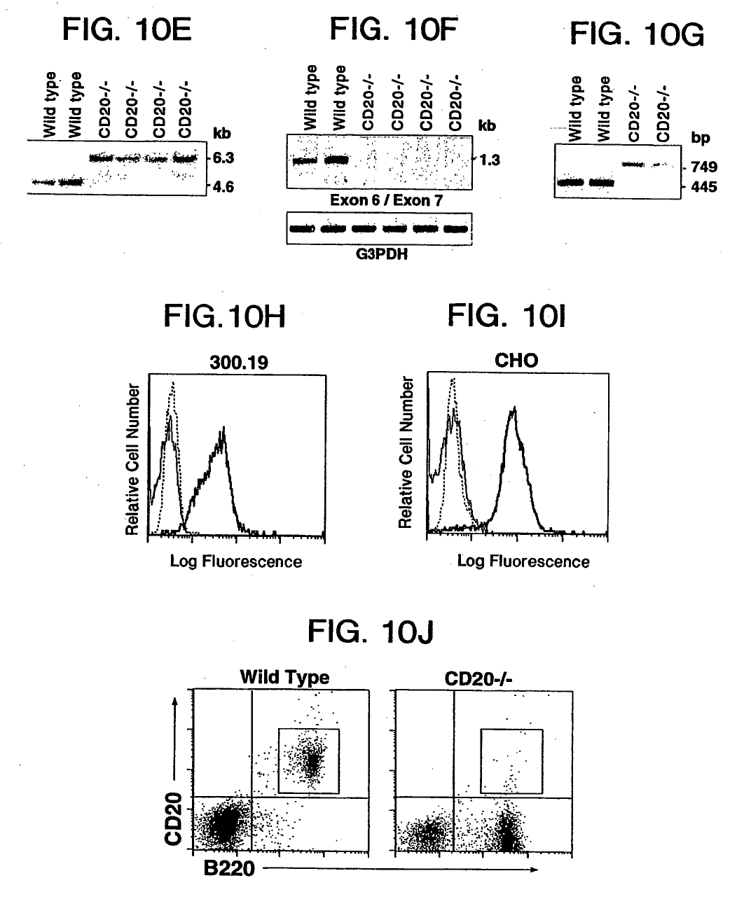

CD20-deficient mice were generated by targeted disruption of the CD20 gene in embryonic stem (ES) cells using homologous recombination, as described in Example 6. A targeting vector was generated that replaces exons encoding part of the second extracellular loop, the 4^{th} transmembrane domain, and the large carboxyl-terminal cytoplasmic domain of CD20 with a neomycin resistant gene (FIG. 10A-D). Appropriate gene targeting generates an aberrant CD20 protein truncated at amino acid position 157 and fused with an 88 amino acid protein encoded by the Neo^{r} gene promoter sequence.

After DNA transfections, 6 of 115 Neo-resistant ES cell clones carried the targeted allele as determined by Southern blot analysis of EcoR V digested genomic DNA using a 1.5 kb DNA probe (FIG. 10D). Appropriate targeting was further verified in two clones by Southern analysis of ES cell DNA digested with BamH I (>12 kb fragment was reduced to a 6.5 kb band in targeted cells), Kpn I (7.2 kb became 5.5 kb), and Ssp I (5.6 kb became 7.0 kb) using the same probe. Cells of one ES cell clone were injected into blastocysts that were transferred into foster mothers. Highly chimeric male offspring (80-100% according to coat color) bred with C57BL/6 (B6) females transmitted the mutation to their progeny (FIG. 10E). Mice homozygous for disruption of the CD20 gene were obtained at the expected Mendelian frequency by crossing heterozygous offspring.

Appropriate targeting of the CD20 gene was further verified by PCR analysis of genomic DNA from homozygous offspring (FIG. 10F). Wild type CD20 mRNA was absent in CD20^{-/-} mice as confirmed by PCR amplification of cDNA generated from splenocytes of CD20^{-/-} mice (FIG. 10G). CD20-deficient mice (CD20^{-/-}) thrived and reproduced as well as their wild type littermates and did not present any obvious anatomical or morphological abnormalities during the first year of life.

Absence of cell surface CD20 protein expression in CD20^{-/-} mice was further verified by staining B220⁺ splenocytes with murine anti-CD20 monoclonal antibodies. Hybridomas producing these antibodies were generated using splenocytes from CD20^{-/-} mice that were immunized with CD20-GFP cDNA-transfected 300.19 cells. Ten hybridomas secreted antibodies reactive with 300.19 (FIG. 10H) and CHO (FIG. 101) cells transfected with CD20-GFP cDNA, but not with untransfected CHO or 300.19 cells (Table 6). These antibodies also reacted with CD20 epitopes expressed on the cell surface of B220⁺ splenocytes from wild type mice, but not with splenocytes from CD20^{-/-} mice (FIG. 10J). Therefore, targeted mutation of the CD20 gene abrogated cell surface CD20 protein expression.

TABLE 6
Anti-CD20 Monoclonal Antibodies Generated in CD20^{-/-} Mice^{a}

| | | | Whole Cell ELISA^{a} | | FACS Analysis^{b} | | |
|---|---|---|---|---|---|---|---|
| Ab Name | Clone Name | Isotype | CD20-CHO | CHO | CD20-300.19 | 300.19 | Spleen |
| MB20-1 | MCD20-5 | IgG1, K | + | − | + | − | + |
| MB20-2 | MCD20-61 | IgG1, K | + | − | + | − | ++ |
| MB20-3 | MCD20-86 | IgG3, K | + | − | + | − | ++ |
| MB20-6 | MCD20-223 | IgG2a, K | + | − | + | − | + |
| MB20-7 | MCD20-243 | IgG2b, K | + | − | + | − | + |
| MB20-8 | MCD20-270 | IgG2b, K | + | − | + | − | + |
| MB20-10 | MCD20-388 | IgG2b, K | + | − | + | − | + |
| MB20-11 | MCD20-392 | IgG2a, K | + | − | + | − | + |
| MB20-13 | MCD20-624 | IgG3, K | + | − | + | − | ++ |
| MB20-14 | MCD20-642 | IgG1, K | + | − | + | − | ++ |

^{a}Values represent reactivity of the monoclonal antibody with adherent monolayers of CHO cells either transfected or untransfected with CD20-GFP cDNA as assessed by a cell-based ELISA. The monoclonal antibodies did not react with GFP cDNA-transfected CHO cells.
^{b}Cell surface reactivity of the monoclonal antibody with single cell suspensions of 300.19 cells either transfected or untransfected with CD20-GFP cDNA or spleen cells from wild type mice. Values represent relative indirect immunofluorescence staining intensity as assessed by flow cytometry and shown in figure 10H-J.

VIII.B. B Cell Development and Function in CD20^{-/-} Mice

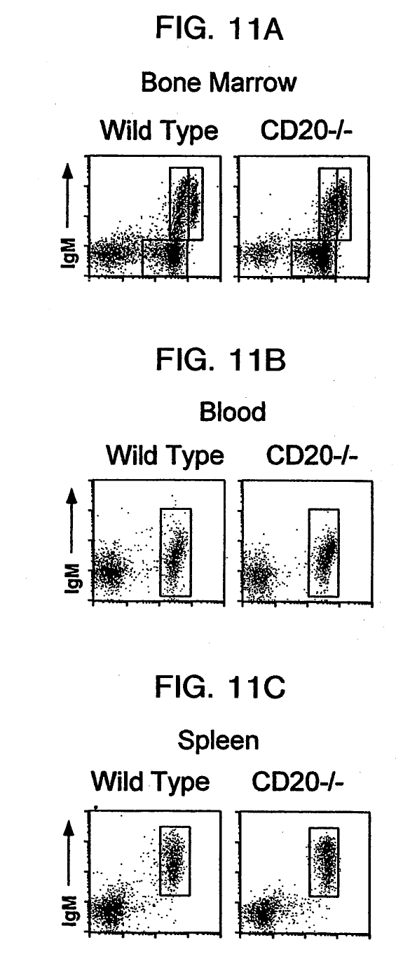

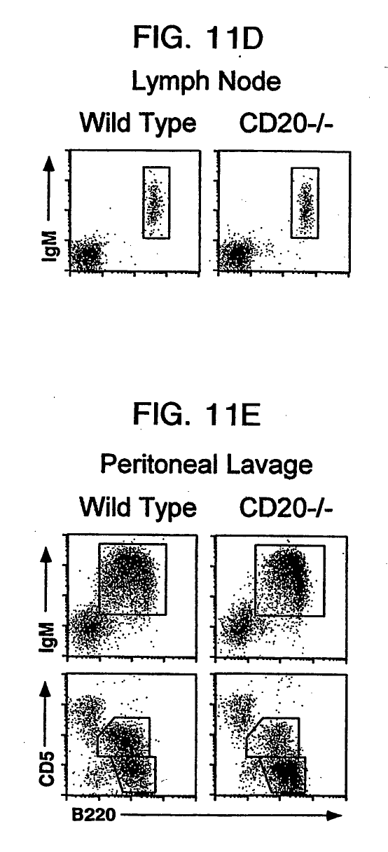

CD20^{-/-} mice did not show an obvious propensity for infections during their first year of life. They had normal frequencies of IgM⁻ B220^{lo} pro/pre-B cells, IgM⁺ B220^{lo} immature B cells and IgM⁺ B220^{hi} mature B cells in the bone marrow (FIG. 11, Table 7). Overall, the number of circulating and spleen IgM⁺ B220⁺ B cells found in CD20^{-/-} mice was increased compared with wild type littermates (Table 7). However, an immunohistochemical analysis of spleen tissue sections revealed a normal architecture and organization of the spleen. In the bone marrow, overall IgM expression was decreased on immature B cells, yet increased on mature B cells when compared with IgM levels expressed by comparable cells in wild type littermates. However, overall IgM expression by mature B220^{hi} B cells in the blood, spleen and lymph nodes was slightly lower in CD20^{-/-} mice (FIG. 11B-D). There were no obvious differences in the size (light scatter properties) of CD20^{-/-} B cells isolated from bone marrow, blood, lymph nodes or spleen when compared with B cells from wild type littermates. These data therefore suggest that CD20 plays a functional role in the development and tissue localization of B cells.

TABLE 7
Frequencies and Numbers of B Lymphocytes in CD20^{-/-} Mice

| | | % of B Lymphocytes | | B cell numbers (×10⁻⁶)^{b} | |
|---|---|---|---|---|---|
| Tissue | Phenotype | Wild Type | CD20^{-/-} | Wild Type | CD20^{-/-} |
| Bone Marrow | B220^{lo}IgM⁻ | 36 ± 2 | 34 ± 3 | | |
| | B220^{lo}IgM⁺ | 19 ± 2 | 13 ± 2* | | |
| | B220^{hi}IgM⁺ | 14 ± 2 | 16 ± 4 | | |
| Blood^{d} | B220⁺IgM⁺ | 61 ± 2 | 60 ± 3 | 3.6 ± 0.5 | 3.9 ± 0.5 |
| Spleen | B220⁺IgM⁺ | 51 ± 6 | 53 ± 5 | 58 ± 12 | 76 ± 7 |
| Lymph Nodes^{e} | B220⁺IgM⁺ | 26 ± 6 | 19 ± 2 | 1.2 ± 0.3 | 0.9 ± 0.1 |
| Peritoneum | B220⁺IgM⁺ | 70 ± 4 | 69 ± 5 | 2.4 ± 0.3 | 3.1 ± 0.4 |
| | B220^{lo}CD5⁺ | 44 ± 4 | 15 ± 5** | 1.5 ± 0.2 | 0.7 ± 0.2** |
| | B220^{hi}CD5⁻ | 28 ± 2 | 59 ± 3** | 1.0 ± 0.1 | 2.7 ± 0.4** |

^{a}Values represent mean (± SEM) results obtained from seven 2-month-old of wild type controls and 10 CD20^{-/-} mice. Numbers represent the percentage of lymphocytes (based on side and forward light scatter properties) expressing the indicated cell surface markers.
^{b}B cell numbers were calculated based on the total number of cells harvested from the indicated tissues.
^{d}The values indicate the number of cells/ml.
^{e}Values represent results from peripheral lymph nodes pairs.
*The percentage or number was significantly different than in wild-type, p < 0.05; ** p < 0.01.

Within the peritoneal cavity, the number of IgM⁺ B220⁺ B cells in CD20^{-/-} mice was similar to that of wild-type littermates (Table 7, FIG. 11E). However, there was a 4-fold decrease in the number of CD5⁺ B220^{lo} B1a cells, with a compensatory increase in the number of CD5⁺ B220^{hi} B2 cells. Therefore, CD20-deficiency predominantly affected the development or clonal expansion of the B1 subpopulation of B cells within the peritoneal cavity. Exemplary methods for quantitating B cell populations are described in Example 7.

VIII.C. Reduced [Ca⁺⁺]i Responses in CD20^{-/-} B Cells

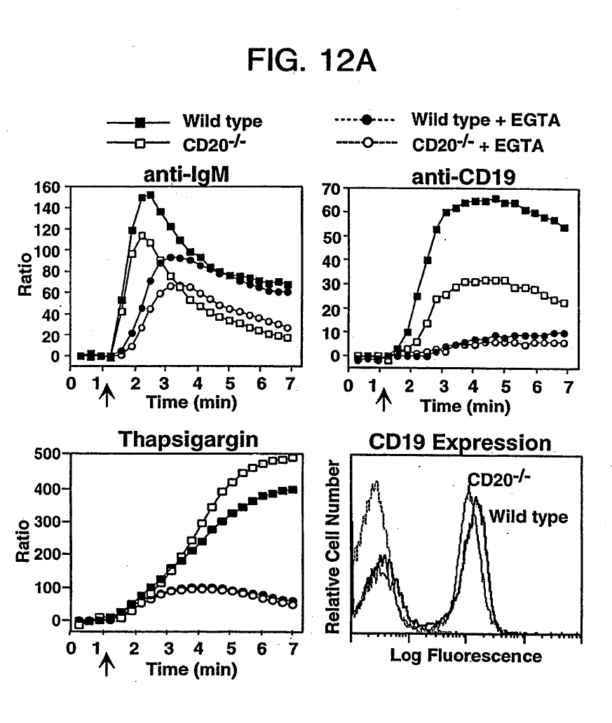

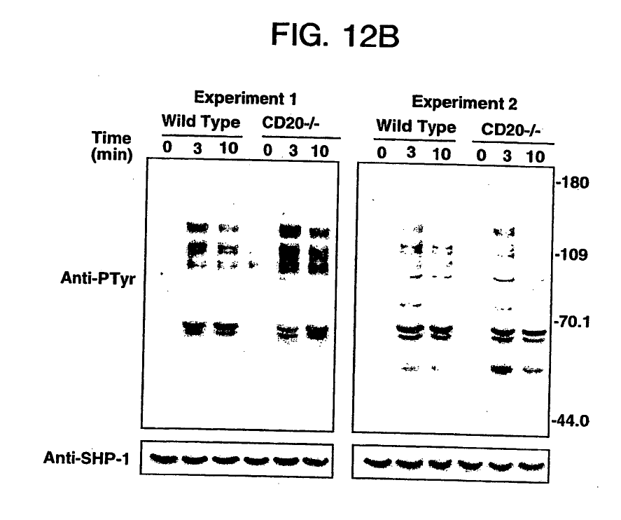

The loss of CD20 significantly altered early B cell signaling responses, measured as described in Example 8. Splenic B220+B cells from CD20^{-/-} mice generated substantially reduced [Ca⁺⁺]i responses following surface IgM ligation when compared with wild type B cells. Decreased [Ca⁺⁺]i responses in CD20^{-/-} B cells were observed in response to both optimal (40 μg/ml, FIG. 12A) and suboptimal concentrations (5 μg/ml) of anti-IgM antibodies. Although the kinetics of [Ca⁺⁺]i responses in CD20^{-/-} B cells was not altered, the magnitude of both the immediate [Ca⁺⁺]i increase and the sustained increase observed at later time points were inhibited by loss of CD20 expression. More dramatic decreases in [Ca⁺⁺]i responses (>50%) by CD20^{-/-} B cells were observed in response to CD19 ligation with optimal concentrations (40 μg/ml) of antibody (FIG. 12A). Reduced [Ca⁺⁺]i responses following CD19 ligation on CD20^{-/-} B cells were likely to result from differences in signaling capacity since Thapsigargin-induced (FIG. 12A) and Ionomycin-induced [Ca⁺⁺]i responses were higher in CD20^{-/-} B cells than in wild type B cells. In addition, CD19 expression levels were not significantly different between CD20^{-/-} and wild type B cells (FIG. 12A).

Chelation of extracellular calcium with EGTA reduced the kinetics and magnitude of the immediate [Ca⁺⁺]i increase observed following IgM crosslinking (FIG. 12A). However, the [Ca⁺⁺]i increase observed at later time points was not substantially inhibited by EGTA treatment. Similar results were observed in CD20^{-/-} B cells. By contrast, chelation of extracellular calcium with EGTA almost eliminated the [Ca⁺⁺]i response observed following CD19 crosslinking (FIG. 12A). This suggests that transmembrane Ca⁺⁺ flux contributes substantially to the [Ca⁺⁺]i responses observed following CD19 crosslinking. That CD20-deficiency had a substantial effect on CD19-induced [Ca⁺⁺]i responses suggests that CD20 can contribute significantly to transmembrane Ca⁺⁺ flux.

The consequences of CD20 loss on transmembrane signal transduction was further evaluated by assessing total cellular protein tyrosine phosphorylation in purified B cells following IgM ligation. Although some variation was observed between B cells from individual mice in individual experiments, overall levels of tyrosine phosphorylation in resting splenic B cells were higher in CD20^{-/-} B cells than in wild type mice (FIG. 12C). In addition, protein phosphorylation in B cells from CD20^{-/-} mice increased more significantly after B cell antigen receptor (BCR) ligation than in wild type B cells. Thus, while CD20 expression can influence BCR-induced tyrosine phosphorylation, decreased [Ca⁺⁺]i responses in CD20^{-/-} B cells are unlikely to result from significant abnormalities in transmembrane signaling through the BCR.

IX. Therapeutic Applications

Another aspect of the present invention is a therapeutic method comprising administering to a subject a substance that modulates MS4A biological activity. Therapeutic substances include but are not limited to chemical compounds, antibodies, and gene therapy vectors. Substances that are discovered by the methods disclosed herein are useful for therapeutic applications related to disorders of MS4A function.

In one embodiment, the present invention provides a method for disrupting MS4A function by immunizing a subject with an effective dose of the disclosed MS4A polypeptide. The immune system of the subject produces an antibody that specifically recognizes the MS4A polypeptide, and binding of the antibody to the MS4A polypeptide abolishes MS4A function.

In another embodiment, the present invention provides MS4A nucleic acid sequences and gene therapy methods for modulating MS4A activity in a target cell. The gene therapy vector can encode a MS4A or sequences encoding a nucleic acid molecule, peptide, or protein that interacts with a MS4A protein.

Vehicles for delivery of a gene therapy vector include but are not limited to a liposome, a cell, and a virus. Preferably, a cell is transformed or transfected with the DNA molecule or is derived from such a transformed or transfected cell. Alternatively, the vehicle is a virus, including a retroviral vector, adenoviral vector or vaccinia virus whose genome has been manipulated in alternative ways so as to render the virus non-pathogenic. Methods for creating such a viral mutation are detailed in U.S. Pat. No. 4,769,331. Exemplary gene therapy methods are also described in U.S. Pat. Nos. 5,279,833; 5,286,634; 5,399,346; 5,646,008; 5,651,964; 5,641,484; and 5,643,567.

The therapeutic methods of the present invention can be applied in the treatment of a variety of conditions, including in the treatment of non-Hodgkin's lymphoma and in the treatment of atopic disorders or other allergenic diseases. Application of the present inventive therapeutic methods are evidenced by the current U.S. Food and Drug Administration approved use of antibodies against CD20 in the treatment of non-Hodgkin's lymphoma. Additionally, the therapeutic methods of the present invention are illustrated in view of the recognition in the art that genetic variations at chromosome 11Q12-13 can also play a role in the pathogenesis of atopic disorders and other allergenic diseases. Indeed, it has been recognized that FcεRIβ contributes to such diseases, and thus the MS4A genes identified in accordance with the present invention are envisioned also to contribute to allergenic disease. Therefore the present therapeutic methods, which pertain to the modulation of the biological activity of an MS4A polypeptide of the present invention have application with respect to the treatment of such disorders.

X. Summary

The invention comprises 19 new genes that are members of a class of genes encoding MS4A proteins. Three members have been described, CD20, FcεRIβ, and HTm4. A gene family has been defined based on a shared chromosomal location, conservation of protein size and structure, gene structure conservation, and similar expression in hematopoietic cells. MS4A proteins function as oligomeric cell surface complexes, and complex assembly using diverse MS4A members is implicated as a mechanism for regulating complex function.

Two members of this class, CD20 and FcεRIβ, have been described functionally, and in each case an important function has been delineated. CD20 is required for cell cycle progression and signal transduction in B lymphocytes. CD20 also regulates Ca⁺⁺ conductance, possibly as a cation channel subunit. Of clinical relevance, antibodies that recognize CD20 are effective in treating non-Hodgkin's lymphoma. FcεRIβ mediates interactions with IgE-bound antigens that lead to degranulation of mast cells, and variation of the FcεRIβ locus is implicated in allergenic disease.

The utility of the MS4A genes is based in part on overlapping or shared functions with known MS4A members. In one case, new MS4A genes have important potential as part of a CD20 complex. The structural description of CD20 complexes suggests that one or more CD20-related proteins constitute the functional complex. Thus, new MS4A proteins can define antigens useful for lymphoma treatment. In another case, MS4A genes are implicated in IgE responses. Atopic disorders (allergy, asthma, eczema, allergic rhinitis) are dysfunctional IgE responses and are associated with a locus on human chromosome 11q containing most members of the MS4A gene family. FcεRIβ is one relevant factor, and recent work supports that FcεRIβ as well as other genetic elements in the region contribute to the disease. Thus, as disclosed herein, the present MS4A sequences also have utility in the characterization, diagnosis, and potential treatment of atopy linked to the chromosomal location wherein MS4A genes are located.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Database Searches and cDNA Isolation

Three hundred and thirty seven nucleotide sequences obtained from the translated GenBank database of expressed sequence tags (ESTs) were assembled into sixty-two subgroups of contiguous linear segments based on their overlapping sequences and potential for encoding proteins homologous with CD20. Based on these subgroups, EST cDNAs (FIG. 1) were obtained from the ATCC and sequenced. Based on the complete sequences of twenty-one near full-length EST cDNAs, eleven novel genes were defined in human and mouse that unified multiple EST subgroups. Near full-length EST clones representing these genes are shown in FIG. 1. These eleven genes and five additional genes were also identified by PCR amplification of transcripts using subgroup-specific primers or primers based on EST sequences. The specific details of how cDNAs representing the five genes that were not identified by EST cDNA clones are indicated below. In all cases, ESTs and cDNAs encoding the predicted coding regions of each putative unique gene were sequenced in both directions and at least two independent ESTs and/or cDNAs representing near full-length gene products were sequenced. Thereby, there was independent confirmation of accuracy for all of the sequences reported.

Based on EST subgroup sequences, cDNAs encoding mouse MS4a4B and MS4a4C were isolated by PCR amplification of C57BL/6 mouse spleen cDNA using both Taq and Pfu DNA polymerase. Primers for MS4a4B (SEQ ID NOs:63-64) amplified an 879 bp fragment. Primers for MS4a4C (SEQ ID NOs:65-66) amplified a 794 bp fragment. EST sequences for MS4a4D only encoded the 3′ end of the predicted protein. Since MS4a4D sequences were closely related to MS4a4B and MS4a4C sequences, a sense 5′ primer (SEQ ID NO:67) based on consensus MS4a4B and MS4a4C sequences and a MS4a4D-specific antisense primer (SEQ ID NO:68) were used to amplify a 773 bp fragment from cDNA of C57BL/6 mouse lung.

MS4a6C was initially identified based on one unique EST sequence (AA028258) encoding a mouse protein homologous with the C-terminal end of MS4a6B. MS4a6C cDNAs were isolated by PCR amplification of C57BL/6 mouse bone marrow cDNA using Taq polymerase. A primer based on identical sequences at the 5′ end of the MS4a6B and MS4a6D cDNAs (SEQ ID NO:69) was used in combination with an antisense primer specific for the unique EST sequence (SEQ ID NO:70) to amplify a 787 bp fragment. Sequences from multiple independent PCR-amplified cDNAs were identical. Subsequently, the PCR-generated 5′ end of the near full-length MS4a6C cDNA was found to be identical to an orphan EST subgroup sequence that had not been linked with defined 3′ sequences. Thereby, the EST subgroup sequences verified that the PCR-amplified 5′ end of the MS4a6C cDNAs was appropriate. In addition, the overall MS4a6C sequence was similar to the sequence of MS4a6B cDNAs without interruption. Thus, the MS4a6C cDNA united sequences identical to those found in two non-overlapping CD20-homologous EST subgroups. cDNAs encoding a 473 bp fragment of mouse MS4a3 were amplified from cDNA of C57BL/6 bone marrow as described above. Primers (SEQ ID NOs:71-72) were obtained based on a single thymic cDNA EST sequence (GenBank AA940479) where the corresponding cDNA was not available.

Human MS4A and mouse MS4a cDNA sequences (MS4A1 to MS4A12) (disclosed herein) were used to search the htgs GenBank human genomic database of unfinished human genomic sequences (http://www.ncbi.nlm.nih.gov/blast/) using the BLAST program. Seventeen phase 1 or phase 2 human genomic DNA sequences encoding potential MS4A genes were assembled into groups of contiguous linear segments based on their overlapping sequences. Three EST clones corresponding to partial MS4A6E transcripts were obtained from the ATCC and sequenced completely on both DNA stands.

All PCR-amplified cDNAs were subcloned and sequenced entirely in both directions. Complete sequencing of at least two distinct PCR-generated cDNAs from both Taq and Pfu enzyme was performed in most cases. Differences between cDNA sequences were only noted when multiple cDNA clones generated by both Taq and Pfu polymerases revealed identical differences. In some cases, cDNAs or EST sequences contained potential intronlexon splice sites that delimited structural domains and aligned with the known intronlexon splice sites of CD20 (Tedder et al. (1989b) J Immunol 142:2560-2568). In these cases, potential introns were flanked by consensus splice donor and/or splice acceptor sequences (Aebi & Weissmann (1987) Trends Genet 3:102-107) or were likely to represent splice variants where exons were deleted.

### Example 2

### RNA Isolation and Reverse Transcription-PCR

Reverse transcription-PCR amplification (RT-PCR) was as described previously (Zhou & Tedder, 1995) with minor modifications. Total RNA was extracted from 1-2×10⁷ hematopoietic cell lines using a RNeasy Mini Kit (Qiagen, Inc., Chatsworth, Calif.) according to the manufacturer's instructions. Human hematopoietic cell lines included one pre-B cell line (NALM-6), three B cell lines (BJAB, DAUDI, and SB), four T cell lines (HSB-2, HUT-78, JURKAT, and MOLT15), two myelomonocytic lines (HL60 and U937), and one erythroleukemia cell line (K562). RNA concentrations were determined by UV absorbance. Ten μg of total RNA was reverse transcribed. In some cases, cDNA from any of 8 different human tissues (colon, ovary, blood mononuclear cells, prostate, small intestine, spleen, testes, and thymus; from CLONETECH Laboratories, Inc., Palo Alto, Calif.) was analyzed. RT-PCR amplification was performed using gene-specific primers identical with protein coding regions of the predicted MS4A genes during 35 cycles (94° C. for 1 min, 55° C. for 1.5 min, 72° C. for 1.5 min, followed by extension at 72° C. for 5 min). Following amplification, the PCR products were separated on 1% agarose-ethidium bromide gels and photographed. G3PDH, a housekeeping gene, was also amplified to control for sample to sample variation. RNA amplified without reverse transcription was used as a negative control, and was negative in all cases.

### Example 3

### Recombinant Production of MS4A Protein

For recombinant production of a protein of the invention in a host organism, a nucleotide sequence encoding the protein is inserted into an expression cassette designed for the chosen host and introduced into the host where it is recombinantly produced. The choice of the specific regulatory sequences such as promoter, signal sequence, 5′ and 3′ untranslated sequence, and enhancer appropriate for the chosen host is within the level of ordinary skill in the art. The resultant molecule, containing the individual elements linking in the proper reading frame, is inserted into a vector capable of being transformed into the host cell. Suitable expression vectors and methods for recombinant production of proteins are well known for host organisms such as E. coli, yeast, and insect cells (see, e.g., Lucknow & Summers (1988) Bio/Technol 6:47). Additional suitable expression vectors are baculovirus expression vectors, e.g., those derived from the genome of Autographica californica nuclear polyhedrosis virus (AcMNPV).

Recombinantly produced proteins are isolated and purified using a variety of standard techniques. The actual techniques used varies depending upon the host organism used, whether the protein is designed for secretion, and other such factors. Such techniques are well known to the skilled artisan. See Ausubel et al. (1994).

### Example 4

### Mouse Anti-Mouse CD20 Monoclonal Antibody Production

Hybridomas producing CD20-specific mouse monoclonal antibodies were generated by the fusion of NS-1 myeloma cells with spleen cells from a CD20^{-/-} mouse immunized with a cell line expressing a mouse CD20-GFP fusion protein. The CD20-GFP fusion protein was generated by subcloning a fragment of the pmB1-1 cDNA (from 159 to 1050 bp of SEQ ID NO:39) into the PEGFP-N1 vector (Clonetech Laboratories Inc., Palo Alto, Calif.) to generate an open reading frame encoding the entire CD20 protein with GFP fused to the carboxyl-terminal end. The resulting plasmid was linearized with ApaL I and used to transfect 300.19 cells, a mouse pre-B cell line, and Chinese Hamster Ovary (CHO) cells. Transfection was by Lipofectamine following the manufacturer's instructions (Clonetech Laboratories, Inc.). Transfected cells were selected using GENETICIN™ (1 mg/ml, GIBCOBRL) in RPMI 1640 media (Sigma) for 300.19 cells or H-12 nutrient mixture (GIBCOBRL) for CHO cells. Both media were supplemented with 10% FCS, L-glutamine, streptomycin and penicillin. Transfected cells expressing high levels of CD20-GFP were isolated by fluorescence-based cell sorting.

### Example 5

### In Vitro Binding Assays

Recombinant protein can be obtained, for example, according to the approach described in Example 4 herein above. The protein is immobilized on chips appropriate for ligand binding assays. The protein immobilized on the chip is exposed to sample compound in solution according to methods well known in the art. While the sample compound is in contact with the immobilized protein, measurements capable of detecting protein-ligand interactions are conducted. Measurement techniques include, but are not limited to, SEDLI, Biacore, and FCS, as described above. Compounds found to bind the protein are readily discovered in this approach and are subjected to further characterization.

### Example 6

### Generation of CD20-Deficient Mice

DNA encoding the CD20 gene was isolated from a phage library prepared from 129/Sv strain mouse DNA (FIG. 10A), mapped with restriction endonucleases, and sequenced to identify intronlexon boundaries (FIG. 10B). The targeting vector was constructed using a pBluescript SK (Stratagene, La Jolla, Calif.)-based targeting vector (p594, provided by Dr. David Milstone, Brigham and Women's Hospital, Boston, Mass.). A DNA fragment starting at the Pst I site in CD20 exon 5 through the EcoR V site in exon 6 (˜1.8 kb) was isolated and blunt end ligated into the targeting vector downstream of the pMC1-HSV thymidine kinase gene and upstream of the neomycin resistance marker obtained from pGK-neo poly A (Stratagene) that contained the PGK promoter and poly A signal sequence. An ˜10 kb DNA fragment beginning at the Kpn I site downstream of exon 8 was also isolated and inserted into the targeting vector downstream of the neomycin resistant gene. The plasmid was linearized using a unique Sal I restriction site proximal to the 3′ end of the CD20 gene insert and used to transfect ES cells.

ES cells were transfected with linearized plasmid DNA and selected for G418 resistance as described (Keller and Smithies (1989) Proc Natl Acad Sci USA 886:8932). Genomic DNA from individual selected clones was digested with EcoR V and used for Southern blot analysis along with a radiolabeled ˜1.5 kb DNA probe that was external to the targeting vector (FIG. 10D). A 4.6 kb genomic DNA fragment hybridized with the probe in wild type ES cells or a 6.3 kb fragment in appropriately targeted ES cells (FIG. 1E). Genomic DNA generated by BamH I, Ssc I or Kpn I digestion was also analyzed for appropriate targeting. The Southern blot pattern obtained in all cases was consistent with the appropriate predicted mutation indicating that detrimental recombinations did not occur in the vicinity of the desired homologous recombination. Cells from appropriately targeted ES cell clones were injected into 3.5 day old C57BL/6 blastocysts that were transferred into foster mothers. Offspring carrying the mutant CD20 allele were identified by Southern blot analysis of DNA obtained from tail biopsies.

High chimeric males (80-100% according to color) were bred with C57BL/6 (B6) females to generate heterozygous offspring with germline gene transmission, which were crossed to generate the homozygous CD20^{-/-} and wild type littermates used for this study. In some cases, B6/129F1J (Jackson Laboratory) were used as controls. Results obtained using wild type littermates of CD20^{+/-} mice were similar and were therefore pooled. All mice were between 2-3 months of age when used for this study. Mice were housed in a specific pathogen-free barrier-facility. All studies and procedures were approved by the Animal Care and Use Committee of Duke University.

### Example 7

### Flow Cytometric Analysis of Lymphocyte Subsets

Single cell suspensions of lymphocytes from the spleen, bone marrow, peripheral lymph nodes, and peritoneal cavity were isolated from CD20^{-/-} and wild type mice and counted using a hemocytometer prior to two-color immunofluorescence analysis. Retroorbital venous plexus puncture was utilized to obtain circulating leukocytes. Leukocytes (0.5×10⁶) were stained at 4° C. using predetermined optimal concentrations of the test monoclonal antibody for 20 min as described (Zhou et al. (1994) Mol Cell Biol 14:3884-3894). Blood erythrocytes Were lysed after staining using the Coulter Whole Blood Immuno-Lyse kit as detailed by the manufacturer (Coulter, Inc., Miami, Fla.). Cells were washed and analyzed on a FACScan flow cytometer (Becton Dickinson, San Jose, Calif.).

Antibodies used in this study included the following: biotin, FITC-conjugated anti-B220 Mab (CD45RA, RA-3, 6B2, provided by Dr. Robert Coffman, DNAXCORP, Palo, Alto, Calif.); PE-conjugated anti-mouse Thy1.2 (Caltag Laboratories, Burlingame, Calif.); B220-PE (Caltag Laboratories, Burlingame, Calif.); biotin-conjugated anti-1-A (BD PharMingen, Franklin Lakes, N.J.); PE or APC-conjugated anti-CD5 (BD PharMingen); PE-conjugated goat anti-mouse IgG3-specific antibody (Southern Biotechnology Associates Inc., Birmingham, Ala.); and biotin-conjugated anti-mouse IgD (Southern Biotechnology Associates Inc., Birmingham, Ala.). FITC or biotin-conjugated goat anti-mouse IgM isotype-specific antibodies (Southern Biotechnology Associates Inc., Birmingham, Ala.) were also used.

Phycoerythrin-conjugated Streptavidin (Southern Biotechnology Associates Inc., Birmingham, Ala.) was used to reveal biotin-coupled monoclonal antibody staining. The percent positively stained lymphocytes was determined using a FACScan flow cytometer (Becton Dickinson, San Jose, Calif.). Positive and negative populations of cells were determined by using unreactive monoclonal antibody (Caltag Laboratories, Burlingame, Calif.) as controls for background staining. Background levels of staining were delineated using gates positioned to include 98% of the control cells. Ten thousand cells with the forward and side light scatter properties of lymphocytes were analyzed for each sample.

### Example 8

### Intracellular Calcium Measurements

Changes in lymphocyte [Ca²⁺]ᵢ, levels were monitored by flow cytometry analysis as described (Fujimoto et al. (1999) Immunity 11:191). Single cell suspension of splenocytes were resuspended (1×10⁷/ml) in RPMI 1640 medium containing 5% FBS, 10 mM HEPES and loaded with 1 μM of indo-1-AM for 30 min at 37° C. Splenocytes were then washed and incubated with a predetermined optimal concentration of FITC-conjugated anti-B220 monoclonal antibody for 15 min at room temperature. The splenocytes were washed again and resuspended at 2×10⁶/ml in medium. The fluorescence ratio (405/525 nm) of B220⁺ splenic B cells was monitored by flow cytometry at baseline for 1 min and for 6 min after stimulation with optimal and suboptimal concentrations of goat F(ab′)₂ anti-IgM antibody (5-40 μg/ml), optimal concentrations of anti-mouse CD19 monoclonal antibody (40 μg/ml), Thapsigargin. (1 μg/ml; Sigma), or Ionomycin (2.67 μg/ml; Calbiochem Biosciences, Inc., La Jolla, Calif.). In some cases, EGTA (5 mM final; pH 7.0) was added to the cells, immediately followed by stimulation with the inducing agents described above. Results were plotted as the fluorescence ratio at 20 sec intervals with background fluorescence subtracted. An increase in the fluorescence ratio indicates an increase in [Ca²⁺]ᵢ.

### Example 9

### Characterization of a MS4A Promoter Region

A preferred in vitro technique for evaluating MS4A promoter function is a transient transfection assay. According to this method, one or more chimeric reporter genes comprising a MS4A promoter region is introduced into a relevant host cell (e.g., a hematopoietic cell), and the resulting level of reporter gene expression is quantitated. Representative methods for making an expression system comprising a promoter region operably linked to a heterologous reporter sequence are disclosed in U.S. Pat. No. 6,087,111.

To analyze the function of a MS4A promoter region in vivo, transgenic mice bearing a chimeric gene comprising a MS4A promoter region are generated, and a level of reporter gene expression in each mouse is determined.

Within a candidate promoter region or response element, the presence of regulatory proteins bound to a nucleic acid sequence can be detected using a variety of methods well known to those skilled in the art (Ausubel et al., 1992). Briefly, in vivo footprinting assays demonstrate protection of DNA sequences from chemical and enzymatic modification within living or permeabilized cells. Similarly, in vitro footprinting assays show protection of DNA sequences from chemical or enzymatic modification using protein extracts. Nitrocellulose filter-binding assays and gel electrophoresis mobility shift assays (EMSAs) track the presence of radiolabeled regulatory DNA elements based on provision of candidate transcription factors. Computer analysis programs, for example TFSEARCH version 1.3 (Yutaka Akiyama: “TFSEARCH: Searching Transcription Factor Binding Sites”, http://www.rwcp.or.jp/papia/), can also be used to locate consensus sequences of known cis-regulatory elements within a genomic region.

### REFERENCES

The publications and other materials listed below and/or set forth in the text above to illuminate the background of the invention, and in particular cases, to provide additional details respecting the practice, are incorporated herein by reference. Materials used herein include but are not limited to the following listed references.
Adelman et al., (1983) DNA 2:183-193.
Adra et al. (1994) Proc Natl Acad Sci USA 91:10178-10182.
Adra et al. (1999) Clin Genet 55:431-437.
Aebi and Weissmann (1987) Trends Genet 3:102-107.
Alam & Cook (1990) Anal Biochem 188:245-254.
Altschul et al. (1990) J Mol Biol 215:403-410.
Altschul et al. (1997) Nucleic Acids Res 25:3389-3402.
Anderson et al. (1984) Blood 63:1424.
Ausubel et al. (1992) Current Protocols in Molecular Biology, John Wylie and Sons, Inc., New York, N.Y.
Barton (1998) Acta Crystallogr D Biol Crystallogr 54:1139-1146.
Batzer et al. (1991) Nucleic Acids Res 19:3619-3623.
Blank et al. (1989) Nature 337:187-189.
Bodanszky, et al. (1976) Peptide Synthesis, John Wiley and Sons, Second Edition, New York, N.Y.
Bubien et al. J Cell Biol 121:1121-1132.
Conner et al. (1983) Proc Natl Acad Sci USA 80:278-282.
Cubitt et al. (1995) Trends Biochem Sci 20:448-455.
Dietrich et al. (1996) Nature 380:149-152.
Dombrowicz et al. (1998) Immunity 8:517-529.
Einfeld et al. (1988) EMBO J. 7:711-717.
Fujimoto et al. (1999) Immunity 11:191.
Furumoto et al. (2000) Biochem Biophys Res Com 273:765-771.
Glover, ed. (1985) DNA Cloning: A Practical Approach, MRL Press, Ltd., Oxford, United Kingdom.
Gorman et al. (1996) Immunity 5:241-252.
Henikoff et al. (2000) Electrophoresis 21(9):1700-1706.
Henikoff & Henikoff (1989) Proc Natl Acad Sci USA 89:10915.
Henikoff & Henikoff (2000) Adv Protein Chem 54:73-97.
Harlow & Lane (1988) Antibodies: A Laboratory Manual, Cold Spring Harbor Laboratory Press, Cold Spring Harbor, N.Y.
Huang et al. (2000) Pac Symp Biocomput 230-241.
Hupp et al. (1989) J Immunol 143:3787-3791.
Hutchens & Yip (1993) Rapid Commun Mass Spectrom 7: 576-580.
Kanzaki et al. (1997a) J Biol Chem 272:14733-14739.
Kanzaki et al. (1997b) J Biol Chem 272:4964-4969.
Kanzaki et al. (1995) J Biol Chem 270:13099-13104.
Karlin & Altschul (1993) Proc Natl Acad Sci USA 90:5873-87.
Kinet (1999) Annu Rev Immunol 17:931-972.
Kinet et al. (1988) Proc Natl Acad Sci USA 85:6483-6487.
Keller & Smithies (1989) Proc Natl Acad Sci USA 886:8932.
Kozak (1986) Cell 44:283-292.
Küster et al. (1992) J Biol Chem 267:12782-12787.
Kyte et al. (1982) J Mol Biol 157:105.
Lander & Botstein (1989) Genetics 121:185-199.
Landgren et al. (1988) Science 241:1007.
Landgren et al. (1988) Science 242:229-237.
Latorra et al. (1994) PCR Methods Appl 3(6):351-358.
Li & Herskowitz (1993) Science 262:1870-1874.
Liedberg et al. (1983) Sensors Actuators 4:299-304.
Lin et al. (1996) Cell 85:985-995.
Luckow & Schutz (1987) Nucleic Acids Res 15:5490.
Luo et al. (1996) Biotechniques 20(4):564-568.
Luyckx et al. (1999) Proc Natl Acad Sci USA 96(21):12174-12179.
Madge et al. (1972) Phys Rev Lett 29:705-708.
McLaughlin et al. (1998) Oncology 12:1763-1769.
Maiti et al. (1997) Proc Natl Acad Sci USA, 94:11753-11757.
Malmquist (1993) Nature 361:186-187.
Mohan et al. (1999) 1999 103:1685-1695.
Needleman & Wunsch (1970) J Mol Biol 48:443-453.
Ohtsuka et al. (1985) J Biol Chem 260:2605-2608.
Onrust et al. (1989) J Biol Chem 264:15323-15327.
Pearson & Lipman (1988) Proc Natl Acad Sci USA 85: 2444-2448.
Postic et al. (1999) J Biol Chem 275(1):305-315.
Ra et al. (1989) Nature 19:1771-1777.
Rose & Botstein (1983) Meth Enzymol 101:167-180.
Rossolini et al. (1994) Mol Cell Probes 8:91-98.
Saiki et al. (1985) Bio/Technology 3:1008-1012.
Sambrook et al. eds. (1989) Molecular Cloning A Laboratory Manual, Cold Spring Harbor Laboratory Press, Cold Spring Harbor, N.Y.
Sauer (1998) Methods 14(4):381-392.
Saqi et al. (1999) Bioinformatics 15:521-522.
Schalkwyk et al. (1999) Genome Res 9:878-887.
Sieghart et al. (1999) Neurochem Int 34:379-385.
Silhavy et al. (1984) Experiments with Gene Fusions, Cold Spring Harbor Laboratory, Cold Spring Harbor, N.Y.
Singh et al. (1989) Biotechniques 7:252-261.
Smith & Waterman (1981) Adv Appl Math 2:482.
Stamenkovic & Seed (1988) J Exp Med 167:1975-1980.
Stashenko et al. (1980) J Immunol 125:1678-1685.
Tedder & Engel (1994) Immunol Today 15:450454.
Tedder et al. (1988a) J Immunol 141:4388-4394.
Tedder et al (1988b) Proc Natl Acad Sci USA 85:208-212.
Tedder et al. (1989a) J Immunol 142:2555-2559.
Tedder et al. (1989b) J Immunol 142:2560-2568.
Tijssen (1993) Laboratory Techniques in Biochemistry and Molecular Biology-Hybridization with Nucleic Acid Probes, part I chapter 2, Elsevier, New York, N.Y.
U.S. Pat. No. 4,196,265
U.S. Pat. No. 4,554,101
U.S. Pat. No. 4,736,866
U.S. Pat. No. 5,162,215
U.S. Pat. No. 5,234,933
U.S. Pat. No. 5,260,203
U.S. Pat. No. 5,326,902
U.S. Pat. No. 5,489,742
U.S. Pat. No. 5,550,316
U.S. Pat. No. 5,573,933
U.S. Pat. No. 5,614,396
U.S. Pat. No. 5,625,125
U.S. Pat. No. 5,648,061
U.S. Pat. No. 5,741,957
U.S. Pat. No. 6,087,111
Vidal et al. (1996) Proc Natl Acad Sci USA 93(19):10315-10320.
Weiner (1999) Semin Oncol 26:43-51.
Whiting (1999) Neurochem Int 34:387-390.
WO 93/25521
WO 97/47763
Worrall et al. (1998) Anal Biochem 70:750-756.
Zhou et al. (1994) Mol Cell Biol 14:3884-3894.
Zhou & Tedder (1995) Blood 86:3295-3301.
Zimmer et al. (1993) Peptides, pp. 393-394, ESCOM Science Publishers, B.V.

It will be understood that various details of the invention can be changed without departing from the scope of the invention. Furthermore, the foregoing description is for the purpose of illustration only, and not for the purpose of limitation—the invention being defined by the claims.

What is claimed is:

1. An isolated MS4A polypeptide, or functional portion thereof, comprising:
(a) a polypeptide encoded by the nucleotide sequence of any one of the odd-numbered SEQ ID NOs:1-37;
(b) a polypeptide encoded by a nucleic acid molecule that is substantially identical to any one of the odd-numbered SEQ ID NOs:1-37;
(c) a polypeptide having the amino acid sequence of any one of the even-numbered SEQ ID NOs:2-38;
(d) a polypeptide that is a biological equivalent of the polypeptide of any one the even-numbered SEQ ID NOs:2-38; or
(e) a polypeptide which is immunologically cross-reactive with an antibody that shows specific binding with a polypeptide of any one of the even-numbered SEQ ID NOs:2-38.

2. An isolated nucleic acid molecule encoding a MS4A polypeptide, comprising:
(a) the nucleotide sequence of any one of the odd-numbered SEQ ID NOs:1-37; or
(b) a nucleic acid molecule substantially identical to any one of the odd-numbered SEQ ID NOs:1-37.

3. The isolated nucleic acid molecule of claim 2, comprising a 20 nucleotide sequence that is identical to a contiguous 20 nucleotide sequence of any one of the odd-numbered SEQ ID NOs:1-37.

4. A chimeric gene, comprising the nucleic acid molecule of claim 2 operably linked to a heterologous promoter.

5. A vector comprising the chimeric gene of claim 4.

6. A host cell comprising the chimeric gene of claim 4.

7. The host cell of claim 6, wherein the cell is selected from the group consisting of a bacterial cell, a hamster cell, a mouse cell, and a human cell.

8. A method of detecting a nucleic acid molecule that encodes a MS4A polypeptide, the method comprising:
(a) procuring a biological sample comprising nucleic acid material;
(b) hybridizing the nucleic acid molecule of claim 2 under stringent hybridization conditions to the biological sample of (a), thereby forming a duplex structure between the nucleic acid of claim 2 and a nucleic acid within the biological sample; and
(c) detecting the duplex structure of (b), whereby a MS4A nucleic acid molecule is detected.

9. An antibody that specifically recognizes a MS4A polypeptide of claim 1.

10. A method for producing an antibody that specifically recognizes a MS4A polypeptide, the method comprising:
(a) recombinantly or synthetically producing a MS4A polypeptide, or portion thereof;
(b) formulating the polypeptide of (a) whereby it is an effective immunogen;
(c) administering to an animal the formulation of (b) to generate an immune response in the animal comprising production of antibodies, wherein antibodies are present in the blood serum of the animal; and
(d) collecting the blood serum from the animal of (c), the blood serum comprising antibodies that specifically recognize a MS4A polypeptide.

11. A method for detecting a level of MS4A polypeptide, the method comprising
(a) obtaining a biological sample comprising peptidic material; and
(b) detecting a MS4A polypeptide in the biological sample of (a) by immunochemical reaction with the antibody of claim 9, whereby an amount of MS4A polypeptide in a sample is determined.

12. A method for identifying a substance that modulates MS4A function, the method comprising:
(a) isolating a MS4A polypeptide of claim 1;
(b) exposing the isolated MS4A polypeptide to a plurality of substances;
(c) assaying binding of a substance to the isolated MS4A polypeptide; and
(d) selecting a substance that demonstrates specific binding to the isolated MS4A polypeptide.

13. A method for modulating MS4A function in a subject, the method comprising:
(a) preparing a pharmaceutical composition, comprising a substance identified according to the method of claim 10 or 12, and a carrier; and
(b) administering an effective dose of the pharmaceutical composition to a subject, whereby MS4A activity is altered in the subject.

14. The method of claim 13, wherein the substance is an antibody, a protein, a peptide, or a chemical compound.

15. The method of claim 13, wherein MS4A activity is regulation of the abundance of target cell subpopulations.

16. The method of claim 13, wherein MS4A activity is regulation of [Ca²⁺]ᵢ levels.

17. A method for identifying a candidate compound as a modulator of MS4A gene expression, the method comprising:
(a) exposing a cell sample with a candidate compound to be tested, the cell sample containing at least one cell containing a DNA construct comprising a modulatable transcriptional regulatory sequence of a MS4A-encoding nucleic acid and a reporter gene which is capable of producing a detectable signal;
(b) evaluating an amount of signal produced in relation to a control sample; and
(c) identifying a candidate compound as a modulator of MS4A gene expression based on the amount of signal produced in relation to a control sample.

18. The method of claim 17, wherein the modulatable transcriptional regulatory sequence of a MS4A-encoding nucleic acid comprises a sequence that is immediately upstream of the initial coding region of a MS4A gene as set forth in any one of SEQ ID NOs:73-81.

19. A method for modulating MS4A function in a subject, the method comprising:
(a) preparing a gene therapy vector having a nucleotide sequence encoding a MS4A polypeptide or a nucleotide sequence encoding a nucleic acid molecule, peptide, or protein that interacts with a MS4A nucleic acid or polypeptide; and
(b) administering the gene therapy vector to a subject, whereby the function of MS4A in the subject is modulated.
